(19) United States
(12) Patent Application Publication
(10) Pub. No.: US 2021/0275692 A1
(43) Pub. Date: Sep. 9, 2021

(54) SOLUTION COMPRISING FLUORESCENT DYE AS FIDUCIAL MARKER

(71) Applicants: DANMARKS TEKNISKE UNIVERSITET, Kgs. Lyngby (DK); NANOVI A/S, KGS. LYNGBY (DK)

(72) Inventors: Jonas Rosager HENRIKSEN, ALLERØD (DK); Thomas Lars ANDRESEN, VANLØSE (DK); Anders Elias HANSEN, LINHAMN (SE); Andreas Tue Ingemann JENSEN, VIRUM (DK); Linda Maria BRUUN, COPENHAGEN S (DK); Rasmus Irming JØLCK, KGS. LYNGBY (DK)

(21) Appl. No.: 17/254,220

(22) PCT Filed: Jun. 19, 2019
(86) PCT No.: PCT/EP2019/066205

§ 371 Date: Dec. 18, 2020

(30) Foreign Application Priority Data
Jun. 19, 2018 (EP) 18178519.7
Nov. 5, 2018 (EP) 18204336.4

(51) Int. Cl. A61K 49/00; A61K 51/04; A61K 51/12

(52) U.S. Cl. CPC A61K 49/0032; A61K 49/006; A61K 49/0052; A61K 51/1213; A61K 49/0073; A61K 51/0491; A61K 51/121; A61K 49/0071

(57) ABSTRACT

The present disclosure relates to a solution comprising a water insoluble carbohydrate and a fluorescent dye, such as a near infrared (NIR) contrast agent, wherein the solution sets under aqueous conditions, such as in vivo, to form e.g. a gel, a glass, a semi-solid, a solid, a crystal or any mixtures thereof. The disclosure further relates to preparation of such solution and use of such solution for in vivo imaging and/or guidance of surgery or interventional therapeutic procedures.

### TECHNICAL FIELD

The present disclosure relates to a solution comprising a water insoluble carbohydrate and a fluorescent dye, such as a near infrared (NIR) contrast agent, wherein the solution sets under aqueous conditions, such as in vivo, to form e.g. a gel, a glass, a semi-solid, a solid, a crystal or any mixtures thereof. The disclosure further relates to preparation of such solution and use of such solution for in vivo imaging and/or guidance of surgery and/or interventional procedures.

### BACKGROUND

Surgery has long been the cornerstone in the treatment of solid cancers. The extent of surgery, the surgical approach and its successful outcome vary according to the type of cancer, its stage, size, distribution and location. Surgery performed in the early stages of cancer results in good treatment outcomes. The objective of the surgical procedure can be palliative or radical. Palliative surgery aims to relieve the symptoms caused by cancer and radical surgery has a curative intend. Surgery is sometimes also done with the aim of preventing cancer as in case of resection of colorectal cancer precursors or resection of ground glass opacities in the lung. For cancer surgery with a curative and preventive intend it is paramount that all malignant cells are removed from the patient. Therefore, precision of the surgical procedure becomes paramount.

Thorough planning of the surgical procedure is needed when the target is not visible for the surgeons naked eye. For planning purposes pre-operative scans (most often MRI and CT) are used to build 3D models of target structures and organs. Volumetric reconstructions from pre-operative scans are also the basis for the development of patient-specific virtual reality simulations, through which the surgeon can perform procedural training before carrying out the actual intervention.

Another approach being utilized more and more is the use of real-time image guidance of the surgical procedures. Technology such as the C-arm and intraoperative cone-beam computed tomography (CBCT) are being integrated in hybrid operating rooms. The same applies to fluoroscopy and magnetic resonance imaging (MRI). Both the C-arm and the CBCT are however based on x-ray based imaging, in which soft tissue is not visible and where gold tissue markers are often used as a companion. Ultrasound is sometimes also used for intraoperative guidance—with and without use of markers—as in the case of breast cancer.

In lung cancer, screening programs are emerging and increasing numbers of small-sized solitary pulmonary nodules (SPNs) are identified at an early stage. Optimally, such SPNs should be surgically removed by video assisted thoracic surgery (VATS) at the time of diagnosis to prevent progression of the disease. This situation has induced a frustrating problem for both surgeons and patients, as the majority of the SPNs are non-palpable due to their small size and/or distance from the pleura, rendering them impossible for the surgeon to locate and remove. Consequently, the VATS procedure is delayed until the nodule has grown to a palpable size. Such delays are unsatisfactory and increase the risk of progression to metastatic cancer, which significantly worsens prognosis and increases treatment associated costs.

Modern mammography identifies lesions at increasingly smaller sizes which is challenging for surgeons to accurately locate and excise. A number of approaches are currently applied to improve the surgical outcome, including wire-guided (WGL) and radio-guided occult lesion localization (ROLL). The observed benefit of surgical guidance has expanded the use of ROLL techniques to solitary lung tumors and thyroid carcinomas.

To overcome the issue of identifying the cancer lesion during surgery, markers that can be positioned and identified during surgery are therefore intensively explored to fully exploit the potential of diagnostic images during the surgical disclosure. Examples of tested markers are: (i) tissue colour stains such as Methylene Blue and near infrared

(NIR) dyes such as ICG, (ii) Lipiodol, a radiopaque oil, (iii) Hook wires, (iv) gold fiducials and (v) ⁹⁹mTc radiolabelled markers. The optimal marker is: i) visible on the diagnostic images, ii) easy to locate during surgery at any tissue depth, iii) does not delocalize or migrate after placement, iv) can be positioned with high precision, and v) does not cause additional risks to the patient, such as pneumothorax or other unnecessary complications. However, none of the currently available markers fulfil these criteria.

NIR-camaras, SPECT scanners and gamma probe detetors are being integrated both in standard surgery as well as in robotic surgical systems, such as the da Vinci system from Intuitive Surgical. The same applies to PET scanners. Robotic-assisted surgery uses robotic arms to perform laparoscopic procedures. The advantages of using robotic surgery include greater visualization, enhanced dexterity and greater precision, which for the patient leads to a number of benefits including reduced pain and discomfort, faster recovery time and return to normal activities, smaller incisions, resulting in reduced risk of infection and minimal scarring.

There is therefore an urgent need in the field for development of good fiducial markers to use with the developing technologies in the field of image-guided surgery.

### SUMMARY

The present disclosure provides excellent fiducial markers for guiding surgical interventions or marking of sites in the body, such as after biopsies. The present disclosure provides an injectable solution that sets under aqueous conditions, such as in vivo, to form e.g. a gel, a glass, a semi-solid, a solid, a crystal or any mixtures thereof, after which it may provide a system for controlled release or retention of fluorescent dyes and/or acts as a tissue marker for imaging by one or multiple imaging modalities. The solution of the present disclosure joins modern biomaterial and dye technology into new surgical markers that are biocompatible, degradable, may be visible on multiple image modalities and that are readily injectable, compatible with state-of-the-art bronchoscopes. Other advantages of the solution of the present disclosure includes that it is compatible with conventional syringe and needle injection as well as state-of-the-art injection technologies, including but not limited to; endoscopes, CT and US guided aspiration and injection technology. The solution of the present disclosure thus provides deposition of fluorescent dyes at defined positions e.g. at the site of a tumour, foreign body, critical margins and nerves.

The present disclosure relates to a solution comprising a water insoluble carbohydrate, a fluorescent dye and a solvent having a log P in the range of −2 to 2. In one aspect, the fluorescent dye has a log P above 2. The hydrophobicity of the fluorescent dye ensures that the diffusion rate in the solution of the fluorescent dye is low and/or that its affinity for the aqueous phase is low and thereby the fluorescent dye is retained in the deposited solution. Upon administration of the solution to e.g. a site of a tumour, the solvent of the solution diffuses into the surrounding environment, resulting in an increase in viscosity and ultimately setting of the solution to form e.g. a gel, a glass, a semi-solid, a solid, a crystal or any mixtures thereof, thereby providing a kinetic trap of the fluorescent dye.

The fluorescent dye is thereby retained at the administered site of injection or direct administration e.g. dispersion in the surgical bed after tumor removal, allowing precise positioning of the fiducial marker. In a second alternative aspect, the fluorescent dye is covalently conjugated to polyethylene glycol (PEG) and has a molecular weight above 2000. The hydrophilic nature of PEG provides release of the fluorescent dye from the solution, after which the fluorescent dye-PEG conjugate may enter the regional lymphatic system, stain this and accumulate in lymph nodes, when administered to an individual in need thereof.

Benefits of the gel based fiducial marker of the present disclosure include that the gels are less prone to migration or spreading caused by diffusion compared current standard procedures, can accommodate multiple imaging modalities, such as NIR/PET/SPECT/CT markers, do not need surgical removal after ended treatment, have improved biocompatibility and can be inserted/injected using minimally invasive application methods. Overall this leads to improved patient comfort and treatment outcome, and the easy injectability though small gauge needles or bronchoscopes expands the possible indications where fiducial marker is relevant.

As described herein above, good fiducial markers are needed in the field of image-guided surgery. The solution of the present disclosure can accommodate multiple imaging modalities, such as NIR/PET/SPECT/CT/MRI/US markers and can be used with either PET or SPECT imaging, or handheld gamma probe detection. Currently, no liquid fiducial marker technology capable of retaining radioactivity at the site of injection is available, and less attractive methods are employed where a solution of ^{99m}Tc labelled macro-aggregates diluted with a CT contrast medium is injected into the nodule by CT guidance. Such solutions however suffer from rapid clearance of the marker and spreading of activity which lowers precision and usability.

An ideal marker should be visible on diagnostic images and easy to identify during a VATS procedure. The current disclosure describes multimodal fiducial markers that: i) are easy to inject in the diseased tissue using unguided injections, ultrasound (US), computed tomography (CT) or fluoroscopy image guidance of injections, and ii) will improve the probability of locating e.g. even small sized nodules situated deep within the lung tissue far from the pleura surface, foreign bodies in soft tissue, tumor margins, critical structures and post-surgical beds where additional tissue is requested to be removed. These markers are fluids before injection and are compatible with state-of-the-art electromagnetic navigation bronchoscopes (ENB), which enables placement of the marker with high precision. Upon injection, the solution sets to form a gel, a glass, a semi-solid, a solid, a crystal or any combination thereof, which minimizes the risk of migration and enables the surgeon to identify the lesion by palpation for peripherally located SPNs.

The solution of the present disclosure may be used for all surgical procedures/indications, where fiducial markers are warranted for guidance. Robotic surgery is another field of application of the current disclosure where guidance by imaging allows the robot to navigate using diagnostic images as roadmaps and fiducial makers inside the patient as beacons.

Thus, in one aspect, the present disclosure relates to a solution comprising

a. a water insoluble carbohydrate,

b. a fluorescent dye, and

c. an organic solvent having a log P in the range of −2 to 2.

In one aspect, the present disclosure relates to a solution as disclosed herein wherein the fluorescent dye has a log P above 2, thereby providing retention of the fluorescent dye in the solution under aqueous conditions.

In one aspect, the present disclosure relates to a solution as disclosed herein wherein the fluorescent dye is covalently conjugated to polyethylene glycol and has a molecular weight above 2000 Da, thereby providing release of the fluorescent dye from the solution under aqueous conditions.

Such release of fluorescent dye-PEG conjugate may provide accumulation of said conjugate in the lymph nodes following release from the solution in vivo.

In one aspect, the present disclosure relates to a solution as described herein, wherein the fluorescent dye is coordinated to a radionuclide. Coordination of the fluorescent dye to a radionuclide may provide a fiducial marker detectable by multiple imaging modalities and nuclear medical detection technologies.

In one aspect, the present disclosure relates to a solution as described herein, for use as an in vivo imaging tool.

In another aspect, the present disclosure relates to a method of in vivo imaging, the method comprising
a. administering a solution as described herein to an individual in need thereof,
b. excitation of the fluorescent dye, and
c. detection of the fluorescent dye.

In one aspect, the present disclosure relates to use of the solution as described herein for in vivo imaging.

In one aspect, the present disclosure relates to use of the solution as described herein for guidance of surgery and interventional therapeutic procedures.

### DESCRIPTION OF DRAWINGS

FIG. 1: Absorbance and fluorescence spectra of PC1, PC2 and PC3 dissolved in toluene or marker formulations. (A) PC1 dissolved in toluene and (B) in SAIB:xSAIB:EtOH 70:10:20 marker formulation. (C) PC2 dissolved in toluene and (D) in SAIB:xSAIB:EtOH 70:10:20 marker formulation. (E) PC3 dissolved in toluene and (F) in SAIB:BA 80:20 marker formulation.

FIG. 2: Fluorescence self-quenching analysis for the phthalocyanine dye PC2 in SAIB:x-SAIB:EtOH 70:10:20. (A) Fluorescence emission spectra of PC2 given as function of the dye concentration. (B) Normalized maximum fluorescence intensity for PC2 obtained in (A) given as function of the PC2 dye concentration. The emission spectra were recorded in triplicates by excitation at 768 nm. (C) Surface fluorescence intensity images recorded with (+EtOH) and without EtOH (−EtOH) for a range of different PC2 dye concentrations. (D) Normalized surface fluorescence intensity for PC2 obtained from (C) given as function of the PC2 dye concentration. The surface fluorescence image intensities were obtained by intensity analysis using ImageJ.

FIG. 3: In vitro release of PC2 dye from a SAIB:xSAIB:EtOH 70:10:20 formulation. UVvis spectra of PC2 in the PBS release media on day 6 after injection into buffer (conducted in triplicate). A standard corresponding to 10% release was included for reference. Nearly none of the PC2 dye was released within a timeframe of 6 days.

FIG. 4: Copper induced quenching of PC2 dye in SAIB:xSAIB:EtOH 70:10:20 investigated by UVvis and fluorescence. (A) Normalized absorption spectra of PC2 given as function of Cu/PC2 ratios. (B) Normalized fluorescence intensities of PC2 given as function of the Cu/PC2 ratio.

FIG. 5: Fluorescence emission of SSIB-Cy7.5 when formulated in SAIB:xSAIB:EtOH 70:10:20 or LOIB:xSAIB:EtOH:D&Cv2 70:10:20:0.1. (A) Fluorescence emission of SSIB-Cy7.5 in SAIB:xSAIB:EtOH 70:10:20 marker formulation for different SSIB-Cy7.5 dye concentrations. (B) Normalized absorbance and emission of LOIB:xSAIB:EtOH:D&Cv2:SSIB-Cy7.5 70:10:20:0.1:0.01.

FIG. 6: Surgical resection of SSIB-Cy7.5 NIR markers (LOIB:xSAIB:EtOH:D&Cv2:SSIB-Cy7.5 70:10:20:0.1:0.01) from the thigh and testicle of a male rat. RGB (A) and NIR (B) image of surgical resection from the right thigh of a rat. RGB (C) and NIR (D) image of marker inside the testicle of a rat (NIR camera integration time 40 ms).

FIG. 7: Injection of SSIB-Cy7.5 marker (LOIB:xSAIB:EtOH:D&Cv2:SSIB-Cy7.5 70:10:20:0.1:0.01) in porcine lung tissue. (A) Opened thoracic cavity (B) Injection of 100 μL SSIB-Cy7.5 markers at three positions. (C) RGB image of marker positions and tissue depth (left=deep, middle=medium, right=surface). (D) NIR image of the three markers (83 ms integration time). Dashed circles are inserted in C and D to highlight the position of the three markers.

FIG. 8: Radio-TLC chromatograms of SAIB:xSAIB:EtOH:PC2 (70:10:20:0.01) radiolabelled with ⁶⁴Cu. (A) Complex formation of ⁶⁴Cu-PC2 in a SAIB:xSAIB:EtOH marker, which results in Rf=0.9-1.0. (B) Control experiment of radiolabelling of a SAIB:xSAIB:EtOH marker which do not contain PC2 resulting in Rf=0.

FIG. 9: Transfer efficiency and in vitro release of ⁶⁴Cu radiolabelled SAIB:xSAIB:EtOH 70:10:20 marker formulations containing PC2 dye. (A) Transfer efficiency of ⁶⁴Cu radiolabelled SAIB:xSAIB:EtOH 70:10:20 marker formulations given as function of PC2 dye concentration. (B) In vitro release of ⁶⁴Cu into TRIS buffered EDTA liposome containing media given as function of time for SAIB:xSAIB:EtOH 70:10:20 marker formulations containing varying PC2 dye concentrations. All experiments were conducted in triplicates, and the results are reported as average±SEM.

FIG. 10: Change in biodistribution of ⁶⁴Cu, marker volume, and marker fluorescence intensity as function of time post injection. (A) Biodistribution of ⁶⁴Cu in the marker, liver, heart and bladder based on PET. (B) Marker volume given as function of time. (C) Total NIR fluorescence intensity emitted from the marker given as function of time post administration. (D) Biodistribution of ⁶⁴Cu 48 h post injection based on organ well counting data.

FIG. 11: Representative images of one mouse subcutaneously injected with ⁶⁴Cu radiolabelled SAIB:xSAIB:EtOH:PC2 70:10:20:0.01 marker formulation. Coronal PET and CT images are shown 1 h, 24 h and 48 h post injection, whereas the FLI images shows the NIR fluorescence of PC2 1 h, 24 h, 48 h, 2 w, 3 w and 4 w post injection.

FIG. 12: (A) PET/CT/FLI/NIR images of a mice subcutaneously injected with ⁶⁴Cu (8HQ) radiolabelled LOIB:xSAIB:EtOH:D&Cv2:SSIB-Cy7.5 70:10:20:0.1:0.01 marker, and corresponding changes in ⁶⁴Cu biodistribution, NIR fluorescence intensity and marker volume. (B) ⁶⁴Cu biodistribution in the marker, liver and kidney as function of time. (C) Total flux recorded in NIR fluorescence from the marker after 18 h and 44 h post injection. (D) Relative volume change of the marker given as function of time post injection.

FIG. 13: Representative SPECT/CT and FLI/Xray images of one mouse injected subcutaneously with 50 μL ¹²⁵I-radiolabeled LOIB:xSAIB:EtOH:D&Cv2:SSIB-Cy7.5 marker formulation given as function of time. The mouse was scanned and imaged 10 min, 1 week, 2 weeks and 3 weeks post injection.

### DETAILED DESCRIPTION

The present disclosure relates to a solution comprising a fluorescent dye, such as a NIR contrast agent, wherein the solution sets under aqueous conditions, such as in vivo, to form e.g. a gel, a glass, a semi-solid, a solid, a crystal or any mixtures thereof and thereby the solution provides deposition of the fluorescent dye at a defined position e.g. at the site of a tumour.

The solution of the present disclosure may also be used as a fiducial marker, for in vivo imaging and for guidance during surgery.

### Definitions

The term “solution”, as used herein refers to a liquid composition comprising the components of the invention. In one embodiment, all components are dissolved in said liquid composition. In another embodiment, some or all the components are dispersed in said liquid composition, such as to create a colloidal dispersion. The terms “solution” and “dispersion” may be used interchangeably herein.

The term “setting of the solution”, as used herein, refers to a change in the physical properties if the solution, changing from a fluid form to a gel form, a semi-solid form, a solid form, a crystalline form or any combinations thereof. The solution of the present disclosure is in a fluid form until subjected to aqueous conditions, such as in vivo conditions, whereupon the organic solvent diffuses into the surrounding environment, resulting in setting of the solution to form a gel, a semi-solid, a solid, a crystal or any combination thereof. In other terms, “the solution sets”, to form a gel, a semi-solid, a solid, a crystal or any combination thereof, under aqueous conditions. Thus, when referring to “the set solution”, it is referred to the gel, the semi-solid, the solid, the crystal or any combination thereof, formed by the solution under aqueous conditions. Similarly, when referring to a gel, the gel mixture, the semi-solid, the solid, the crystal or any combination thereof, it is referred to the composition or depot resulting from subjecting the solution of the invention to aqueous conditions. “The form of the set solution” may thus be a gel, a semi-solid, a solid, a crystal or any combination thereof. The terms “the set solution”, “gel” and “depot” may be used herein interchangeably.

The term “log P”, as used herein, refers to the partitioning coefficient of a given compound between a water phase and a 1-octanol phase. Log P is given as the logarithm of the ratio of concentrations of the given compound in the water and the 1-octanol phase. Log P is a measure of the difference in solubility of the compound in these two phases. Positive log P values are generally characteristic of hydrophobic compounds, whereas negative log P values indicate a hydrophilic compound.

The term “aqueous conditions”, as used herein, refers to solutions and/or conditions comprising mainly water. The aqueous conditions may be in vitro conditions, such as a buffer system. Alternatively, the aqueous conditions may be in the human or animal tissue also referred to as in vivo conditions, such as at a tumor site.

The term “comprising” should be understood in an inclusive manner. Hence, by way of example, a composition comprising compound X, may comprise compound X and optionally additional compounds.

The term “Førster resonance energy transfer (FRET)”, also known as fluorescence resonance energy transfer (FRET), resonance energy transfer (RET) or electronic energy transfer (EET), refer to a mechanism describing energy transfer between two light-sensitive molecules (chromophores). A donor chromophore, initially in its electronic excited state, may transfer energy to an acceptor chromophore through nonradiative dipole-dipole coupling. The efficiency of this energy transfer is inversely proportional to the sixth power of the distance between donor and acceptor, making FRET extremely sensitive to small changes in distance.

### Solution

The present disclosure relates to a solution comprising a water insoluble carbohydrate, a fluorescent dye and an organic solvent. Under aqueous conditions, the organic solvent diffuses into the surrounding environment resulting in the setting of the solution to form e.g. a gel, a glass, a semi-solid, a solid, a crystal or any combination thereof.

Thus, in one embodiment, the present disclosure relates to a solution comprising
a. a water insoluble carbohydrate,
b. a fluorescent dye, and
c. an organic solvent having a log P in the range of −2 to 2.

The solutions are sometimes referred to as “markers” herein.

The solution is designed to control the diffusion rate of the fluorescent dye in the solution and/or the set solution. More preferably, the solution is designed to control the diffusion rate of the fluorescent dye out of the solution and/or the set solution. By controlling said diffusion rate, the release or retention of the fluorescent dye from the solution and/or the set solution is controlled. This allows for design of a solution providing controlled release of the fluorescent dye or retention of the fluorescent dye under aqueous conditions.

Administration of the solution of the disclosure may be performed by injection at the site of interest. Thus, in one embodiment, the solution of the present disclosure is injectable by means of an injection needle. The solution of the present disclosure may be a viscous solution. Thus, in one embodiment, the solution has a viscosity in the range of 1-1000 cP, for example in the range of 1-750 cP, such as in the range of 1-500 cP, for example in the range of 1-250, such as in the range of 1-100, for example in the range of 100-1000 cP, such as in the range of 100-500 cP.

The water insoluble carbohydrate of the solution provides the property of the solution to set under aqueous conditions. The hydrophobicity and viscosity of the solution and the set solution can be controlled by the nature of said water insoluble carbohydrate, thereby controlling the diffusion rate of the fluorescent dye.

The set solution of the present disclosure is typically degraded in vivo within 3-12 months. The water insoluble carbohydrates of the solution of the present disclosure are bio-compatible compounds that upon degradation or hydrolysis result in formation of sugars that are well tolerated in tissues, organs etc.

In one aspect, the fluorescent dye of the solution has a log P above 2. A fluorescent dye with a high Log P may be less prone to diffuse out of the deposited solution into an aqueous phase compared to a fluorescent dye with a low Log P. The hydrophobicity of the fluorescent dye ensures that the diffusion rate in the solution of the fluorescent dye is low and/or that its affinity for the aqueous phase is low and thereby the fluorescent dye is retained in the deposited solution. The fluorescent dye is thereby retained at the administered site, allowing precise positioning of the fiducial marker.

In a second aspect, the fluorescent dye of the solution is covalently conjugated to polyethylene glycol (PEG) and has a molecular weight above 2000. The hydrophilic nature of PEG provides release of the fluorescent dye from the solution, after which the fluorescent dye-PEG conjugate may enter the lymphatic system and accumulate in lymph nodes, when administered to an individual in need thereof.

The solvent of the solution serves to dissolve the water insoluble carbohydrate and the fluorescent dye. The solvent should possess the properties of a) being able to dissolve the components of the solution and b) diffuse from the solution into the surrounding environments under aqueous conditions.

In one embodiment, the amount of organic solvent is in the range of 1 to 30%, for example 1 to 20%, such as 1 to 15%, for example 1 to 10%, such as 5 to 10%.

The solution of the present disclosure may in one embodiment further comprise a further solvent, also referred to herein as a co-solvent, such as a monoglyceride, diglyceride and/or triglyceride.

Thus in one embodiment, the present disclosure relates to a solution comprising
a. a water insoluble carbohydrate,
b. a fluorescent dye,
c. an organic solvent having a log P in the range of −2 to 2, and
d. a further solvent, such as a monoglyceride, diglyceride and/or triglyceride.

In one embodiment, the amount of further solvent is in the range of 0 to 50%, such as in the range of 0 to 40%, for example in the range of 0 to 30%, such as in the range of 0 to 20%, for example in the range of 0 to 10%.

The solution of the present disclosure may further comprise an imaging agent. Such imaging agent will allow visualization by imaging modalities other than NIR of the solution once deposited e.g. in vivo.

Thus in one embodiment, the present disclosure relates to a solution comprising
a. a water insoluble carbohydrate,
b. a fluorescent dye,
c. an organic solvent having a log P in the range of −2 to 2, and
d. an imaging agent.

In a separate embodiment, the present disclosure relates to a solution comprising
a. a water insoluble carbohydrate,
b. a fluorescent dye,
c. an organic solvent having a log P in the range of −2 to 2,
d. a further solvent, such as a monoglyceride, diglyceride and/or triglyceride, and
e. an imaging agent.

In one embodiment, the present disclosure relates to a solution comprising
a. a water insoluble carbohydrate according to formula (II) in the range of 40-60 w/w %
b. 2,11,20,29-Tetra-tert-butyl-2,3-naphthalocyanine coordinated to Cu-64 in the range of 0.001-1 w/w %,
c. Ethanol in the range of 15-25 w/w %, and
d. an imaging agent according to formula (III) in the range of 20-40 w/w %.

In one embodiment, the present disclosure relates to a solution providing controlled release of said fluorescent dye.

In a separate embodiment, the present disclosure relates to a solution providing retention of said fluorescent dye.

Water Insoluble Carbohydrate

The water insoluble carbohydrate of the solution provides the property of the solution to set under aqueous conditions. The hydrophobicity and viscosity of the solution and the set solution can be controlled by the nature of said water insoluble carbohydrate, thereby controlling the diffusion rate of the fluorescent dye. Furthermore, the form of the set solution may be varied by varying the water insoluble carbohydrate of the solution.

The term “water insoluble carbohydrate” as used herein refers to a carbohydrate having a log P in the range 2-20, such as in the range of 2-15, for example in the range of 2-10, such as in the range of 2-5, for example in the range of 4-20, such as in the range of 4-15, for example in the range of 4-10. The water insoluble carbohydrate may be any monosaccharide, disaccharide, trisaccharide or oligosaccharide.

In one embodiment, the water insoluble carbohydrate is selected from the group consisting of monosaccharides, disaccharides, trisaccharides and oligosaccharides.

The term “oligosaccharide” as used herein refers to a saccharide polymer comprising up to 10 monosaccharide units, such as up to 9 monosaccharide units, for example up to 8 monosaccharide units, such as up to 7 monosaccharide units, for example up to 6 monosaccharide units, such as up to 5 monosaccharide units, for example up to 4 monosaccharide units. The oligosaccharide may be linear or branched.

In one embodiment, the water insoluble carbohydrate is a monosaccharide selected from the group consisting of Glucosamine, Galactosamine, Mannosamine, Mannose, Rhamnose, Rhamnosamine, Galactose, Allose, Allosamine, Altrose, Altrosamine, Gulose, Gulosamine, Idose, Idosamine, Talose and Talosamine.

The saccharides of the present disclosure may be in either the L- or the D-form. Furthermore, the monosaccharide units of the disaccharides, trisaccharides and the oligosaccharides may be linked by either α or β glycosidic bonds in which α,β anomeric mixtures at any ratio may exist.

In one embodiment, the water insoluble carbohydrate is a disaccharide selected from the group consisting of maltose, trehalose, lactose, sucrose, Galp-(1→2)-Glc, Galp-(1→3)-GlcN, Galp-(1→4)-Glc, Glcp-(1→4)-Glc, Glcp-(1→6)-Glc, Glcp-(1→2)-GlcN, Galp-(1→4)-ManN, Glcp-(1→4)-GalN, Manp-(1→3)-Glc, ManNp-(1→4)-Gal, GalNp-(1→3)-ManN, GlcNp-(1→6)-GalN, Rhamnp-(1→6)-Glc, Glcp-(1→1)-Glcp, Talp-(1→4)-Glu, Glup (1→3)-Ido, GlcNp-(1→4)-GlcN, GlcNp-(1→6)-GlcN.

In one embodiment, the water insoluble carbohydrate is a disaccharide selected from the group consisting of maltose, trehalose, lactose and sucrose.

In one embodiment, the water insoluble carbohydrate is a trisaccharide selected from the group consisting of raffinose, Galp-(1→2)-Glcp-(1→3)-Galp, Galp-(1→4)-Glcp-(1→6)-GlcN, Galp-(1→4)-Glcp-(1→6)-Gal, Glcp-(1→4)-Glcp-(1→4)-Glcp, Glcp-(1→6)-Glcp-(1→6)-Glc, Galp-(1→6)-Glcp (1→2)-Fruf, Glcp-(1→3)-Fruf-(2→1)-Glcp, Galp-(1→4)-ManNp-(1→3)-Glu, Glcp-(1→4)-GalN-(1→2)-Man, Manp-(1→3)-Glcp-(1→4)-GlcN, Man Np-(1→4)-Galp-(1→3)-Glc, GalNp-(1→3)-ManNp-(1→6)-GlcN. Rhamnp-(1→6)-Glcp-(1→4)-GlcN, Galp-(1→6)-Glcp-(1→1)-Glcp, Talp-(1→4)-Glup-(1→2)-Man, Glup (1→3)-Idop-(1→6)-Glu, GlcNp-(1→6)-GlcNp (1→4)-GlcN.

In one embodiment, the water insoluble carbohydrate is raffinose.

In one embodiment, the water insoluble carbohydrate is a oligosaccharide selected from the group consisting of Galp-(1→4)-Glcp-(1→6)-glcp-(1→4)-Glc, Galp-(1→4)-Glcp-(1→4)-Glcp-(1→4)-Glcp-(1→4)-Glc, Galp-(1→4)-Glcp-(1→4)-Galp-(1→4)-Glc, Glcp-(1→4)-Glcp-(1→4)-Glcp-(1→4)-Glc, Galp-(1→6)-Glcp-(1→6)-Galp-(1→6)-Glc, Galp-(1→6)-Glcp-(1→6)-Galp-(1→4)-Glc, Galp-(1→6)-Glcp-(1→6)-Glcp-(1→4)-Glc, GlcNp-(1→4)-GlcNp-(1→6)-GlcNp-(1→4)-GlcN, GlcNp-(1→6)-Galp-(1→6)-Glcp-(1→2)-Fruf, Galp-(1→4)-Glcp-(1→3)-Fruf-(2→1)-Glcp, Talp-(1→4)-Glup-(1→2)-Man-(1→3)-Glu, Glup (1→3)-Idop-(1→6)-Glup-(1→2)-Gal, cellulose acetate, cellulose acetate butyrate and cellulose acetate proprionate.

In one embodiment, the water insoluble carbohydrate comprises one or more hydroxyl groups functionalized to form esters. Such ester may be formed by a bond between the hydroxyl group(s) of the carbohydrate and the carbonyl group of an alkanoyl(s).

In one embodiment, the water insoluble carbohydrate comprises one or more hydroxyl groups functionalized to form C2-C7 esters.

The term “Cn-Cm esters” as used herein refers to ester functional groups formed by a bond between an alcohol and an alkanoyl comprising between n and m carbon atoms. For example a C2-C7 ester is an ester functional group formed by a bond between an alcohol and the carbonyl group of a C2-C7 alkanoyl, an alkanoyl comprising between 2 and 7 carbon atoms.

Thus, in one embodiment, the water insoluble carbohydrate comprises one or more hydroxyl groups functionalized to form esters, wherein the esters are formed by a bond between the hydroxyl group(s) of the carbohydrate and the carbonyl group of an alkanoyl(s).

In one embodiment, the number of hydroxyl groups of the water insoluble carbohydrate functionalized to form esters is n, n-1, n-2, n-3, n-4 or n-5, wherein n is the total number of hydroxyl groups of the carbohydrate.

In one embodiment, all hydroxyl groups of the water insoluble carbohydrate are functionalized to form esters.

In one embodiment, the esters of the water insoluble carbohydrate are C2-C10 esters, such as C2-C9 esters, for example C2-C8 esters, such as C2-C7 esters, for example C2-C6 esters, such as C2-C5 esters, for example C2-C4 esters, such as C2-C3 esters.

In one embodiment, the esters of the water insoluble carbohydrate are C2-C7 esters.

In one embodiment, the alkanoyl is selected from acetyl, propanoyl, butanoyl, isobutanoyl, pentanoyl, hexanoyl, heptanoyl and benzoyl.

In one embodiment the alkanoyl is selected from acetyl, propanoyl, isobutanoyl and benzoyl.

In one embodiment, the water insoluble carbohydrate is selected from the group consisting of maltose octaisobutyrate (MOIB), sucrose diacetate hexaisobutyrate (SAIB), sucrose octaisobutyrate (SOIB), lactose octaisobutyrate (LOIB), trehalose octaisobutyrate (TOIB).

In one embodiment, the water insoluble carbohydrate is selected from the group consisting of sucrose diacetate hexaisobutyrate (SAIB) and lactose octaisobutyrate (LOIB).

The water insoluble carbohydrate may be a mixture of different water insoluble carbohydrate. In embodiment, the water insoluble carbohydrate is a mixture of lactose octaisobutyrate and lactose octabenzoate, or a mixture of lactose octaisobutyrate and sucrose octabenzoate.

In one embodiment, the water insoluble carbohydrate has a structure according to formula (I),

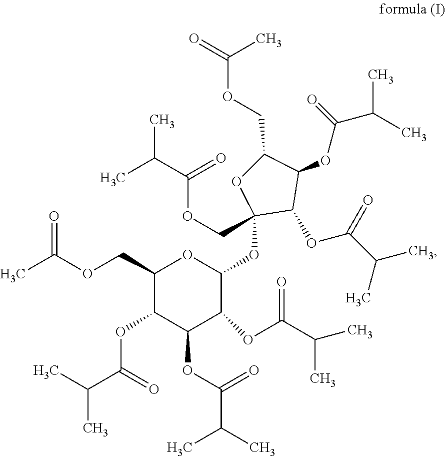 [SMILES: CC(=O)OC[C@H]1O[C@@](COC(=O)C(C)C)(O[C@H]2O[C@H](COC(C)=O)[C@@H](OC(=O)C(C)C)[C@H](OC(=O)C(C)C)[C@H]2OC(=O)C(C)C)[C@@H](OC(=O)C(C)C)[C@@H]1OC(=O)C(C)C]

In one embodiment, the water insoluble carbohydrate has a structure according to formula (II),

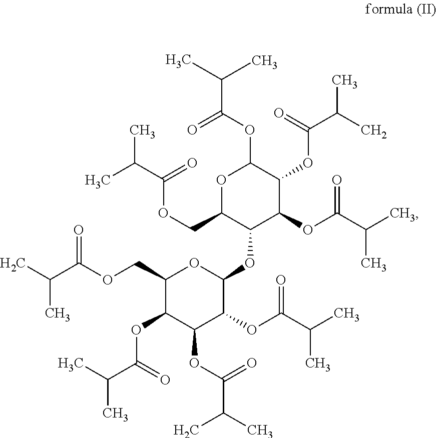

In one embodiment, the water insoluble carbohydrate is raffinose undecaisobutyrate.

Fluorescent Dyes

In one embodiment, the fluorescent dye is selected from the group consisting of rhodamines, BODIPY, Alexa Fluor, NBD, Cyanine dyes (Cy3) and Carboxy-fluorescein.

In one embodiment, the fluorescent dye is a NIR contrast agent.

Conventional and near infrared (NIR) fluorophores are often comprised of highly conjugated molecules characterized of being hydrophobic (log P>0) unless chemically modified to be soluble in aqueous solution by incorporation of charged residues and or hydrophilic polymers such as PEG. The hydrophobic characteristics of such dyes ensure good compatibility with the hydrophobic solutions of the present disclosure and enable high retention in the solution and/or set solution. Such fluorophores are characterized by an excitation and emission spectrum which is separated by a Stoke-shift of varying size. Conventional fluorophores emit photons in the visible spectrum (200-700 nm) range, NIR-1 fluorophores emit photons in the 700-900 nm range, whereas NIR-II fluorophores emit photons in range above 900 nm. The tissue absorption and scattering of photons is however high in the visible spectra range (below 600 nm), and the fluorescence emitted from conventional fluorophores is consequently highly attenuated. Above 600 nm, the tissue absorbance is highly reduced, and scattering of emitted photons are increasingly reduced as function of the photon wavelength. Fluorophores emitting light in the NIR-I and NIR-II region are thus visible at tissue depth of several centimeters, i.e. the excitation and emitted photons can pass several centimeters of tissue, which allows for identification of NIR labeled markers using NIR cameras inside an organ during surgery. Lower levels of autofluorescence from tissues in the NIR-I and NIR-II spectral region also improves the signal to noise ratio enabling better detectability of such dyes in tissues. The NIR-II dyes experience the largest reduction in photon scattering in the tissue, enabling visualization at greater tissue depths and acquisition of more focused (less diffuse) fluorescence signals, which is a major advantage in surgical imaging using fiducial markers.

In one embodiment, two or multiple dyes are incorporated in the solution which may allow for FRET (forster resonance energy transfer). Such inclusion of multiple fluorophores may serve to induce a larger shift between the excitation and emission light.

Current organic fluorophores used as labeling reagents for biomolecule conjugation have limitations in photostability. This compromises their performance in applications that require a photostable fluorescent reporting group. For example, in molecular imaging and single molecule microscopy, photostable fluorescent labels are important for observing and tracking individual molecular events over extended period of time. In the current disclosure, high concentrations of dye may be embedded in the solution which circumvents the issue of photobleaching.

Still, higher photostability of the embedded dyes is warranted as this enables longtime imaging, ensures reproducibility of the solutions performance as NIR marker even after repeated exposure/NIR imaging or when exposed other light sources. Improved storage stability is yet another outcome of higher photostability. Phthalo- and naphthalo-cyanine and porphyrin dyes are optimal hydrophobic NIR dyes for embedding in solutions and exhibit extreme photostability compared to traditional organic dyes. These dyes are about 40 to 125 times more photostable than current near-IR fluorophores, e.g. Alexa Fluor® 680, Cy 5.5, Cy 7 and IRDye™ 800 CW dyes; and about 20 times more photostable than tetramethylrhodamine (TMR), one of the most photostable organic dyes.

In one embodiment the NIR contrast agent is a NIR-I contrast agent.

In one embodiment, the NIR contrast agent is a NIR-II contrast agent.

In one embodiment the NIR contrast agent is selected from the group consisting of Indocyanine green (ICG), Methylene blue (MB), CH1055, IRDye800CW, Non-sulfonated and sulfonated cyanine dyes (Cy5, Cy5.5, Cy7, Cy7.5), Zwitterionic cyanine dyes (ZW800-1), Phosphonated cyanine dyes (Pam78, P800SO3), Quaternary ammonium cyanine dyes (C700-OMe, C800-OMe), BODIPY dyes (mPB, BAP-5), Alexa Fluor dyes (Alexa Fluor 702, Alexa Fluor 749 and Alexa Fluor 790.

In one embodiment the NIR contrast agent is a cyanine dyes selected from the group consisting of Cyanine7.5-alkyne, Cyanine7.5-amine, Cyanine7.5-azide, Cyanine7.5-carboxylic acid, Cyanine7.5-hydrazide, Cyanine7.5-maleimide, Cyanine7.5-NHS ester, Cyanine7.5-tetrazine, Cyanine7-alkyne, Cyanine7-amine, Cyanine7-azide, Cyanine7-carboxylic acid, Cyanine7-hydrazide, Cyanine7-maleimide, Cyanine7-NHS ester, Cyanine7-tetrazine, Cy5-alkyne and Cy5.5-alkyne.

In one embodiment the NIR contrast agent is a cyanine dyes selected from the group consisting of Cyanine7.5-alkyne, Cyanine7.5-amine, Cyanine7.5-azide, Cyanine7.5-carboxylic acid, Cyanine7.5-hydrazide, Cyanine7.5-maleimide, Cyanine7.5-NHS ester and Cyanine7.5-tetrazine.

In one embodiment the NIR contrast agent is a cyanine dyes selected from the group consisting of Cyanine7-alkyne, Cyanine7-amine, Cyanine7-azide, Cyanine7-carboxylic acid, Cyanine7-hydrazide, Cyanine7-maleimide, Cyanine7-NHS ester and Cyanine7-tetrazine.

In one embodiment the NIR contrast agent is a cyanine dyes selected from the group consisting of Cy5-alkyne, Cy5.5-alkyne, Cy7-alkyne and Cy7.5-alkyne.

In one embodiment the NIR contrast agent is selected from the group consisting of porphyrines, phthalocyanines and naphthalocyanines.

In one embodiment the NIR contrast agent is selected from the group consisting of 2,3,7,8,12,13,17,18-Octaethyl-21H,23H-porphine, 5,10,15,20-Tetraphenyl-21H,23H-porphine, 2,9,16,23-Tetra-tert-butyl-29H,31H-phthalocyanine, 1,4,8,11,15,18,22,25-Octabutoxy-29H,31H-phthalocyanine, 2,3,9,10,16,17,23,24-Octakis(octyloxy)-29H,31H-phthalocyanine, 2,11,20,29-Tetra-tert-butyl-2,3-naphthalocyanine, 5,9,14,18,23,27,32,36-Octabutoxy-2,3-naphthalocyanine and Antracocyanine.

In one embodiment the NIR contrast agent is selected from the group consisting of 2,9,16,23-Tetra-tert-butyl-29H,31H-phthalocyanine, 1,4,8,11,15,18,22,25-Octabutoxy-29H,31H-phthalocyanine, 2,3,9,10,16,17,23,24-Octakis(octyloxy)-29H,31H-phthalocyanine and 2,11,20,29-Tetra-tert-butyl-2,3-naphthalocyanine, 5,9,14,18,23,27,32,36-Octabutoxy-2,3-naphthalocyanine.

In one embodiment the NIR contrast agent is selected from the group consisting of IFP1.4, IFP2.0, iRFP713 and miRFP703.

In one embodiment, the NIR contrast agent is selected from the group consisting of IR-780, IR-792, IR-895, IR-140, IR-26/27, IR-1048, IR-1061, NIR-II fluorophore-H1 (3,6-Bis[5-{7-Amino-9,9-bis42-(2-trimethylsilanyl-ethoxycarbonyl)-ethyl]-9H-fluoren-2-yl}-thiophene-2-yl]benzo[1,2-c;4,5-c′]bis[1,2,5]thiadiazole), 1,4,8,11,15,18,22,25-Octabutoxy-29H,31H-phthalocyanine and 5,9,14,18,23,27,32,36-Octabutoxy-2,3-naphthalocyanine.

In one embodiment, the NIR contrast agent is selected from the group consisting of IR-780, IR-792, IR-895, IR-140, IR-26/27, IR-1048 and IR-1061.

In one embodiment, the NIR contrast agent is selected from the group consisting of NIR-II fluorophore-H1,1,4,8,11,15,18,22,25-Octabutoxy-29H,31H-phthalocyanine and 5,9,14,18,23,27,32,36-Octabutoxy-2,3-naphthalocyanine.

In one embodiment, the NIR contrast agent is coordinated to a metal. Such coordination of the NIR contrast agent to a metal may facilitate fine-tuning of the excitation and emission wavelengths of the NIR contrast agent. Examples of NIR contrast agents coordinated to a metal include but are not limited to: Lead(II) phthalocyanine, Manganese(II) phthalocyanine, Cu(II) phthalocyanine, cobalt(II) phthalocyanine, aluminium(III) phthalocyanine chloride, Gallium(III) phthalocyanine chloride, Indium(III) phthalocyanine chloride, Iron(III) phthalocyanine chloride, Manganese(III) phthalocyanine chloride, Nikkel(II) phthalocyanine, Titanyl phthalocyanine, Titanium(IV) phthalocyanine dichloride, Zink(II) phthalocyanine. Vanadyl 3,10,17,24-tetra-tert-butyl-1,8,15,22-tetrakis(dimethylamino)-29H,31H-phthalocyanine, Vanadyl 2,3-naphthalocyanine, Cobalt(II) 2,3-naphthalocyanine, Copper(II) 2,3-naphthalocyanine, Copper(II) 5,9,14,18,23,27,32,36-octabutoxy-2,3-naphthalocyanine, Nickel(II) 5,9,14,18,23,27,32,36-octabutoxy-2,3-naphthalocyanine, Tin(IV) 2,3-naphthalocyanine dichloride and Vanadyl 2,11,20,29-tetra-tert-butyl-2,3-naphthalocyanine.

Thus in one embodiment, the NIR contrast agent is selected from the group consisting of Lead(II) phthalocyanine, Manganese(II) phthalocyanine, Cu(II) phthalocyanine, cobalt(II) phthalocyanine, aluminium(III) phthalocyanine chloride, Gallium(III) phthalocyanine chloride, Indium(III) phthalocyanine chloride, Iron(III) phthalocyanine chloride, Manganese(III) phthalocyanine chloride, Nikkel(II) phthalocyanine, Titanyl phthalocyanine, Titanium(IV) phthalocyanine dichloride, Zink(II) phthalocyanine and Vanadyl 3,10,17,24-tetra-tert-butyl-1,8,15,22-tetrakis(dimethylamino)-29H,31H-phthalocyanine.

In a separate embodiment, the NIR contrast agent is selected from the group consisting of Vanadyl 2,3-naphthalocyanine, Cobalt(II) 2,3-naphthalocyanine, Copper(II) 2,3-naphthalocyanine, Copper(II) 5,9,14,18,23,27,32,36-octabutoxy-2,3-naphthalocyanine, Nickel(II) 5,9,14,18,23,27,32,36-octabutoxy-2,3-naphthalocyanine, Tin(IV) 2,3-naphthalocyanine dichloride and Vanadyl 2,11,20,29-tetra-tert-butyl-2,3-naphthalocyanine.

The solutions according to the present disclosure are based on water insoluble carbohydrates mixed with solvents of varying hydrophobicity. Upon administration of the solution to e.g. a site of a tumor, the solvent of the solution diffuses into the surrounding environment, resulting in an increase in viscosity and ultimately setting of the solution to form e.g. a gel, a glass, a semi-solid, a solid, a crystal or any mixtures thereof, thereby providing a kinetic trap of the solution content. The solution and its content, e.g. a fluorescent dye possibly coordinating a radionuclide are thereby retained at the administered site. In order to kinetically trap fluorophore dyes effectively, the solution has to set whereby its viscosity increases from 100-1000 cP to 100000-1000000 cP or higher for solid depots. According to the Stoke-Einstein relation, such a 1000-fold increase in viscosity leads to a 1000-fold reduction in the diffusion rate in the solution, thereby hindering the dyes from escaping the viscous solution.

Likewise, increasing the molecular cross-section of the diffusing dye additionally reduces the mobility of the dye leading to reduced leaching of dye from the solution. Examples of dyes with increasing molecular cross-sections are given in Table 1, where selected dyes with increasing molecular weight are presented. Alternative strategies for increasing the molecular cross-section with the aim of reducing diffusion rate rely on conjugation of smaller dyes to larger constructs, e.g. polymers such as PLA of PNIPAM.

Thus, in one embodiment, the fluorescent dye is conjugated to a polymer, selected from the group consisting of PNIPAM, cellulose acetate butyrate, cellulose acetate, perfluorocarbons, poloxamer pluronics, polyethylene glycol (PEG), polylactic acid (PLA), poly(lactic-co-glycolic acid) (PLGA), poly(L-lactide) (PLA), poly(glycolide) (PGA), ploy(DL-lactide) (DLPLA), poly(dioxanone) (PDO), poly(DL-lactide-co-L-lactide) (LDLPLA), poly(DL-lactide-co-glycolide) (DLPLG), poly(glycolide-co-trimethylene carbonate) PGA-TMC, poly(L-lactide-co-glycolide) (LPLG) or poly(caprolactone) (PCL).

Alternatively, the fluorescent dye may be conjugated to a water insoluble carbohydrate. The water insoluble carbohydrate may be any water insoluble carbohydrate as defined herein elsewhere. In one embodiment, the fluorescent dye is conjugated to a water insoluble carbohydrate selected from the list comprising SAIB, SSIB, LOIB, trisaccharides, oligosaccharaides and cellulose. In one embodiment, the fluorescent dye is conjugated to a water insoluble carbohydrate selected from the group consisting of SAIB, SSIB, LOIB, trisaccharides, oligosaccharaides and cellulose. In one embodiment, the fluorescent dye Cy7.5 is conjugated to the water insoluble carbohydrate SSIB.

After injection of the solution, before the solution fully sets (5-6 h) and the kinetic trap is effective, the solution constituents (dyes, radionuclides etc) can be thermodynamically stabilized in the solution preventing leakage by choosing dyes that have high affinity for the solution and minimal affinity for the aqueous media. Dyes with high affinity for the solution have log P values above 4, such as above 8, for example above 12, which strengthens the hydrophobic interaction of the solution and the dyes, and additionally minimizes the solubility of the dye in aqueous media.

In one embodiment, the fluorescent dye has a log P above 2, such as above 3, for example above 4, such as above 5, for example above 6, such as above 8, for example above 10, such as above 15.

Slow Diffusing Fluorescent Dyes

Quantum dots (Qdots) are particles that may be kinetically trapped in the solution of the present disclosure due to their size, and or surface functionalization with polymers such as PNIPAM to ensure affinity for the solution and or hindered diffusion caused by polymer entanglement. Quantum dots exist in variant covering both the NIR-I and NIR-II optical range.

Qdots are furthermore an optional solution to circumvent some of the problems of quenching associated with the use of organic fluorophores is the use of quantum dots, which have emerged as alternative biological labels. One unique property of Qdot labels is that the emission wavelength is readily tunable by changing the size or chemical composition of the particles. Compared to conventional organic dyes, Qdots have: a) longer fluorescence lifetime (>10 ns); b) much sharper, well-separated emission peaks; c) efficient excitation by a single UV or visible light source; and d) bright fluorescence. Most importantly, quantum dots exhibit remarkable photostability, which is the greatest limitation for organic dyes. Quantitative measurements indicate that Qdots are about 100 times more stable than rhodamine 6G against photobleaching. The total number of photons emitted by a single Qdot before undergoing photobleaching is estimated to be one to two orders of magnitude higher than a typical organic dye molecule.

Another alternative strategy for utilizing the kinetic trap constituted by the solution presented in the current disclosure, is the use of particles or rods with increased cross-sectional area compared to smaller dyes such as the cyanine dyes Cy5, Cy7.5 etc. Such particles have reduced diffusion rates in viscous media due an increased drag force. Examples of such embodiments are metallic or polymeric nanoparticles of size 1-1000 μm functionalized with fluorescent dyes, such as cyanine dyes. Low affinity dyes may alternatively be entrapped inside such particles for improved retention in the solution.

Yet an alternative strategy is utilizing intrinsic fluorescent particles or rods such as quantum dots or carbon nanotubes. Both quantum dots and carbon nanotubes have high quantum yields and span the optical range from NIR-I to NIR-II, i.e. 500-1600 nm.

Thus in one embodiment, the fluorescent dye of the solution as described herein, is selected from the list consisting of quantum dots, nanoparticles and carbon nanotubes.

In one embodiment, the fluorescent dye is a quantumdot selected from the group consisting of CdTe, CdHgTe CdTe/ZnS, CdTe/CdSe, CdSeTe/CdS, CdTe/CdS/ZnS, PbS, PbS/CdS, PbS/CdS/ZnS, InAs/ZnS, InAs/ZnSe, InAs/InP/ZnSe, InAsxP₁₋ₓ/InP/ZnSe, CuInS2/ZnS, (CuInSeₓS₂₋ₓ)/ZnS, Ag₂S Ag2Se and Si.

In one embodiment, the fluorescent dye is a rare-earth nanoparticle selected from the group consisting of NaYF₄: Er, Ho, Tm, Pr (host:dopant); NaGdF₄: Nd, Yb, Tm; SrF₂: Nd LaF₃:Nd; LiYF₄: Nd NaY_{0.78}Yb_{0.2}Er_{0.02}F₄.

In one embodiment, the fluorescent dye is a metal nanoclusters selected from the group consisting of Au, Ag or Cu nanoclusters.

In one embodiment, the fluorescent dye is a carbon nanotube, such as a single-walled carbon nanotube.

Organic Solvent

The organic solvent of the solution of the present disclosure serves the roles of dissolving the components of the solution, e.g. dissolving the water insoluble carbohydrate and the fluorescent dye. When the fluorecent dye is provided as a particle, the solvent disperses the particles. Furthermore, the organic solvent should have some miscibility with water, thereby having the propensity for partitioning between the solution and aqueous phase.

Thus, in one embodiment, the organic solvent has a log P in the range of −2 to 2, for example in the range of −1.8 to 1.8, such as in the range of −1.5 to 1.5, for example in the range of −1 to 1, such as in the range of −2 to 1, for example in the range of −1.5 to 1, for example in the range of −1 to 2, such as in the range of −1 to 1.5.

In one embodiment the organic solvent is an alcohol.

In one embodiment, the organic solvent is a C1-C7 alcohol, such as a C1-C6 alcohol, for example a C1-C5 alcohol, such as a C1-C4 alcohol.

The term Cn-Cm alcohol as used herein refers to an alcohol having between n and m carbon atoms. For example, the term C1-C4 alcohol refers to an alcohol having between 1 and 4 carbon atoms.

In one embodiment, the organic solvent is selected from the list consisting of methanol, ethanol, propanol, isopropanol, butanol, isobutanol, tert-butanol, benzyl alcohol, propylene carbonate and dimethyl sulfoxide.

In one embodiment, the organic solvent is selected from the list consisting of ethanol, benzyl alcohol, propylene carbonate and dimethyl sulfoxide.

In one embodiment, the amount of organic solvent in the solution of the present disclosure is in the range of 1 to 30%, for example 1 to 20%, such as 1 to 15%, for example 1 to 10%, such as 5 to 10%.

Further Solvents

The solution of the present disclosure may comprise a further solvent. The further solvents are also referred to as co-solvents as herein described. Further solvents may include but are not limited to monoglycerides, diglycerides and/or triglycerides.

Inclusion of further solvents in the solution may provide means for tuning the release rate of the fluorescent dye. In one embodiment, an increased amount of further solvent results in increased release of the fluorescent dye from the solution and/or set solution.

In one embodiment, the further solvent is a triglyceride, selected from the group consisting of glyceryl tridecanoate (GTD), glyceryl trioctanoate (GTO) and glyceryl trihexanoate (GTH).

In one embodiment, the amount of further solvent is in the range of 0 to 50%, such as in the range of 0 to 40%, for example in the range of 0 to 30%, such as in the range of 0 to 20%, for example in the range of 0 to 10%.

Imaging Modalities

Some embodiments of the disclosure contain fluorescent dyes, such as NIR contrast agents, that are chelators. Such dyes are multifunctional as they allow visualization of the solutions using e.g. NIR fluorescence equipment, but in addition enable complexation of radionuclides. Such solutions may be visible both by NIR cameras but also in PET or SPECT images by embedding radionuclides in the solution via the hydrophobic NIR-chelator. Examples of such NIR-chelators are naphthalo and phthalocyanine derived dyes, porphyrine derived dyes such as texaphyrin.

Upon chelation of a metal cation, the spectral properties of the NIR-chelator dye change, which may be utilized for modifying the optical properties of the fiducial marker, or for detection of special cations present in the tissue after injection.

Application of such solutions that are both visible by NIR and PET imaging allows the surgeon to visualize the fiducial marker after injection via PET, e.g. prior to surgery, and to identify the fiducial marker during the surgical disclosure using a NIR camera. Incorporation of other radionuclide types further allows for SPECT imaging or handheld gamma-probe guidance during surgery. Application of such solutions, allows the surgeon to locate the fiducial marker at greater tissue depths using a gamma-probe and still visualize the fiducial marker at intermediate tissue depth using NIR imaging.

Additionally, radiopague contrast agents such as iodinated carbohydrate esters, iodinated polymers or gold nanoparticles may furthermore be included in the solution which allows for CT imaging or guidance by fluoroscopy during the surgical procedure. The solution of the present disclosure is furthermore visible in magnetic resonance imaging (MRI) due to the intrinsic low water content of the material and by ultrasound (US) due to the higher viscosity and/or ductility compared to tissue.

The solution of the present disclosure, functioning as a multimodal fiducial marker with visibility in NIR/PET/SPECT/CT/MRI and US are highly warranted as it provides possibilities for bridging/alignment of several image modalities when used as common reference points in these. The solutions of the present disclosure are furthermore easy to inject, can be traced realtime on US or fluoroscopy during injection/implantation and following enable the surgeon to identify/locate difficult to reach targets during surgery using gamma-probe detectors at large tissue depth, or NIR imaging on short to medium tissue depths. PET and SPECT imaging of such markers may also be utilized in the surgical procedure, either for verification that the marker actually marks the position of the deceased tissue/lesion or for realtime SPECT guided surgery.

Thus, in one embodiment, the solution comprises a further imaging agent.

Imaging modalities include, but are not limited to, X-ray imaging, CT imaging, MRI, PET imaging, single photon emission computed tomography (SPECT) imaging, nuclear scintigraphy imaging, ultrasonography imaging and/or ultrasonic imaging.

In one embodiment, the further imaging agent is selected from the group consisting of X-ray agent, CT agent, MRI agent, PET agent and SPECT agent.

In one embodiment, the fluorescent dye is coordinated to a radionuclide.

In one embodiment, the radionuclide is selected from the group consisting of Tc-99m, In-111, Ga-67, Lu-177, Tl-201, Sn-117m, Cu-64, Mn-52, Zr-89, Co-55, Sc-44, Ti-45, Sc-43, Cu-61, As-72, Te-152, F-18, Ga-68, C-11, Nd-140 and Te-149.

In one embodiment, the radionuclide is selected from the group consisting of Tc-99m, In-111, Ga-67, Lu-177, Tl-201 and Sn-117m.

In one embodiment, the radionuclide is selected from the group consisting of Cu-64, Mn-52, Zr-89, Co-55, Sc-44, Ti-45, Sc-43, Cu-61, As-72 and Te-152.

In one embodiment, the radionuclide is selected from the group consisting of Cu-67, Cu-64, Mn-52, Zr-89, Co-55, Sc-44, Ti-45, Sc-43, Cu-61, As-72 and Te-152.

In one embodiment, the solution may provide imaging by PET and/or SPECT imaging due to embedding of relevant radionuclides in the solution, e.g. Tc-99m, In-111, Ga-67, Lu-177, Tl-201, Sn-117m, Cu-64, Mn-52, Zr-89, Co-55, Sc-44, Ti-45, Sc-43, Cu-61, As-72, Te-152, F-18, Ga-68, C-11, Nd-140, Te-149.

In one embodiment, the solution may comprise radiohalogenated water insoluble carbohydrates, such as radioiodinated or radiofluorinated water insoluble carbohydrates. Thus in one embodiment, the solution may comprise water insoluble carbohydrates which are labeled with ¹³¹I, ¹²⁵I and/or ¹⁸F. Such labeling may allow visualization of the solution by PET and/or SPECT.

In one embodiment the further imaging agent is an X-ray agent. X-ray agents may comprise one or more iodinated polymers, iodinated oligomers, iodinated lipids, iodinated saccharides, iodinated disaccharides, iodinated polysaccharides, iodinated peptides, or a derivative or a combination thereof. Preferred imaging agents are iodinated compounds such as polymers or sugar molecules such as derivatives of glucose or sucrose or derivatives of disaccharides, trisaccharides or oligosaccharides. The X-ray agent may alternatively be a solid particle comprising, or consisting of, one or more X-ray imaging agents, i.e., compounds that are able to block or attenuate X-ray radiation. Such compounds include transition metals, rare earth metals, alkali metals, alkali earth metals, other metals, as defined by the periodic table.

In one embodiment, the X-ray imaging agents are selected from Iodine (I), gold (Au), Palladium (Pd), Silver (Ag), bismuth (Bi), gadolinium (Gd), iron (Fe), barium (Ba), calcium (Ca) and magnesium (Mg).

In one embodiment, the further imaging agent has a structure according to formula (III),

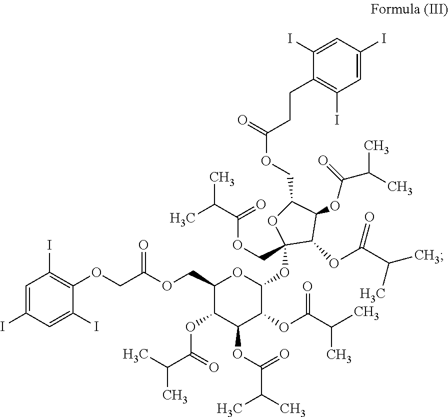 [SMILES: CC(C)C(=O)OC[C@@]1(O[C@H]2O[C@H](COC(=O)COc3c(I)cc(I)cc3I)[C@@H](OC(=O)C(C)C)[C@H](OC(=O)C(C)C)[C@H]2OC(=O)C(C)C)O[C@H](COC(=O)CCc2c(I)cc(I)cc2I)[C@@H](OC(=O)C(C)C)[C@@H]1OC(=O)C(C)C]

In one embodiment, the solution comprises an iodinated derivate of a water insoluble carbohydrate doped into a solution comprising the same class of non-iodinated water insoluble carbohydrate.

In one embodiment, the solution may further comprise a paramagnetic compound for use in imaging modalities such as MRI.

In one embodiment, the solution may provide imaging by MRI with negative contrast due to the negligible water content of the solution.

In one embodiment, the solution may be visible on ultrasound (US) due to its higher viscosity and/or ductility compared to tissue.

In another embodiment, the solution further comprises one or more gasses encapsulated in lipid, polymer or inorganic based particles for ultrasonography imaging. Said gasses may comprise air, sulphur halides such as sulphur hexafluoride or disulphur decafluoride; fluorocarbons such as perfluorocarbons; fluorinated (e.g. perfluorinated) ketones such as peril uoroacetone; and fluorinated (e.g. perfluorinated) ethers such as perfluorodiethyl ether.

Physics of the Solution

The solution of the present disclosure comprises water insoluble carbohydrates and solvents with polar to nonpolar characteristics that together form a solution or dispersion with viscosity in the range 100-1000 cP. In some embodiments of the disclosure, the water insoluble carbohydrates have log P values in the range of 4-10 whereas the solvents used have log P in the range −2-2. When injected into aqueous media or tissue containing interstitial fluid, the solutions undergo a non-solvent induced phase separation (NIPS) where the solvent of the solution partitions into the surrounding aqueous phase leading to precipitation of the water insoluble carbohydrate and formation of a depot with material properties reflecting the carbohydrate. Upon NIPS, some carbohydrate form highly viscous liquids (100000-1000000 cP), e.g. SAIB, while other carbohydrates form brittle or ductile solids, e.g. Maltose octaisobutyrate, Sucrose octaisobutyrate, Lactose octaisobutyrate, Trehalose octaisobutyrate or Raffinose undecaisobutyrate. The brittle and hard carbohydrates can be characterized as being either amorphous, crystalline, glassy-states or mixtures thereof. Examples of such embodiments are solutions comprising SAIB:EtOH 80:20, SAIB:xSAIB:EtOH 50:30:20, LOIB:EtOH 80:20 or LOIB:xSAIB:EtOH 50:30:20, optionally including other carbohydrates, solvents and variations thereof.

In one embodiment, the organic solvent diffuses out of the solution under aqueous conditions, providing a gel, a glass, a semi-solid, a solid, a crystal or any combination thereof.

In one embodiment, the viscosity of the solution increases by more than 1000 centipose (cP) under aqueous conditions, such as more than 5000 cP, for example more than 10000, such as more than 50000 cP, for example more than 100000 cP.

In one embodiment, the viscosity of the set solution is in the range of 100000-1000000 cP, such as in the range of 100000-750000, for example in the range of 100000-500000 cP, such as in the range of 100000-250000 cP.

In one embodiment, the viscosity of the set solution is in the range of 100000-1000000 cP, such as in the range of 250000-1000000 cP, for example in the range of 500000-1000000 cP, such as in the range of 750000-1000000 cP.

Both in depots that are a highly viscous liquids and in depots that are brittle, ductile, amorphous, crystalline or glassy-states, entrapped fluorescent dyes are kinetically trapped as diffusion is impeded by the high viscosity or solid characteristics of the depot. For larger fluorescent dyes or nanoparticles, diffusion inside the solution is reduced even further due to enhanced drag/friction leading to improved effectiveness of the kinetic trapping of these in the set solution.

In some embodiment, co-solvents characterized by log P values in the range 4-10, such as but not limited to mono, di and triglycerides are included in the solution as described herein. When injected into aqueous media or tissue containing interstitial fluids, such solutions undergo NIPS causing the carbohydrate co-solvent solution to form a depot, with tunable viscosities reflecting the carbohydrate material properties, the co-solvent viscosity and the carbohydrate to co-solvent ratio. Examples of such embodiments are SAIB:GTH:EtOH 64:28:8, SAIB:GTO:EtOH 83:9:8 SAIB:xSAIB:GTO:EtOH 66:9:17:8, LOIB:GTO:EtOH 64:28:8, LOIB:xSAIB:GTO:EtOH 50:9:33:8 or variations thereof.

Solvent efflux kinetics from such solutions upon injection into aqueous media or tissues containing interstitial fluids, depends on the aqueous solubility of the solvent in the solution, as well as the solvents affinity for the water insoluble carbohydrate. Solvent with higher log P leads to slower solvent release and slower increase in viscosity of the carbohydrate depot formed by NIPS. As an example, 95% EtOH efflux is completed within 2 hours in vivo for solutions of SAIB:xSAIB:EtOH 50:20:20, i.e. these solutions sets, undergo NIPS in few hours leading to full setting of the solution within 5-6 hours after injection. After the solution setting period, the viscosity of the fluid deposits or impaired diffusion in the solid depots, hinders escape of fluorescent dye.

In one embodiment, the solution sets under aqueous conditions in less than 10 h, such as less than 8 h, for example less than 6 h, such as less than 5 h, such as less than 4 h, for example less than 3 h, such as less than 2 h.

In some embodiments of the disclosure, solutions with low viscosity and high retention of fluorescent dyes are warranted. By mixing water insoluble carbohydrates with co-solvents lower viscosity is obtained.

In cases wherein the carbohydrate and co-solvent are fully compatible, i.e. have similar log P as e.g. LOIB and GTO or SAIB and GTH, one phase systems are predominantly formed, which are unable to retain fluorescent dyes based on the kinetic trap principle due to the reduced viscosity caused by the presence of the co-solvent. However, choosing less compatible carbohydrate esters and co-solvent can lead to microphase separation (MPS) wherein embedded compounds such as fluorescent dyes may be segregated into one phase or the other. In some embodiment of the disclosure, these phases are non-continuous or not fully percolated structures and diffusion and escape of entrapped compounds is hindered. An example could be liquid-liquid coexistence of droplet inside the set solution retaining the fluorescent dye. An embodiment of the disclosure where minimal release is obtained by inclusion of a co-solvent are solutions composed of SAIB:GTO:EtOH.

In one embodiment, the diffusion rate of the fluorescent dye is such that the fluorescent dye is retained in the solution until the solution has set, thereby kinetically trapping the fluorescent dye in the set solution with no or limited release of the fluorescent dye.

Retention in Solution

Guiding of surgical interventions is currently being used in the clinic by injection of freely diffusing dyes such as methylene blue and indocyanine green (ICG). Both dyes are of NIR-I type, and are used for marking of tissue or for monitoring organ perfusion using NIR camera equipment. However, intra-organ injection of such dyes leads to rapid spreading caused by diffusion, which hampers optimal use of these dyes for surgical guidance. The solution of the present disclosure solves this issue by retention of the fluorescent dye. Upon injection of the solution, the solution sets and either traps the NIR dye kinetically or retains the dye due to high affinity between the dye and solution. In this manner, a high concentration of dye is retained at the site of injection allowing the surgeon to identify the region of interest during surgery.

Due to the physics of the solution as described herein above, the fluorescent dye may be retained in the solution and/or the set solution. This allows for precise and stable positioning of the fluorescent dye at the desired position, with minimal leakage to the surrounding tissue and/or organs.

The retention of the fluorescent dye in the solution and/or the set solution may be controlled by the composition of the solution, such as controlling the viscosity of the solution, the form of the set solution, the hydrophobicity of the fluorescent dye, the size of the fluorescent dye and the hydrophobicity of the solvent and/or of the further solvent.

Furthermore, the retention of the fluorescent dye in the solution and/or the set solution may be controlled by the relative log P values of the different components of the solution, as described herein above in the section ‘Physics of the solution’.

In one embodiment, less than 10% of the fluorescent dye is released from the solution and/or the set solution after 5 h under aqueous conditions, such as less than 5%, for example less than 4%, such as less than 2%.

In one embodiment, less than 10% of the fluorescent dye is released from the solution and/or the set solution after 4 h under aqueous conditions, such as less than 5%, for example less than 4%, such as less than 2%.

In one embodiment, less than 10% of the fluorescent dye is released from the solution and/or the set solution after 3 h under aqueous conditions, such as less than 5%, for example less than 4%, such as less than 2%.

In one embodiment, less than 10% of the fluorescent dye is released from the solution and/or the set solution after 2 h under aqueous conditions, such as less than 5%, for example less than 4%, such as less than 2%.

In one embodiment, the aqueous conditions are in vitro conditions, such as a buffer system.

In one embodiment, the aqueous conditions are in vivo conditions, such as injection at a tumor site.

The low release of the fluorescent dye from the solution of the disclosure may provide more precise and stable positioning of the fiducial marker. Furthermore, the low release of the fluorescent dye from the solution of the disclosure may provide a long duration of labelling of the desired tissue by the fluorescent dye.

Controlled Release from Solution

The solution of the present disclosure may also provide controlled release of the fluorescent dye from the solution and/or the set solution. Controlled release of the fluorescent dye may provide labelling of the draining lymph nodes.

The release rate of the fluorescent dye from the solution and/or the set solution may be controlled by the composition of the solution, such as controlling the viscosity of the solution, the form of the set solution, the hydrophobicity of the fluorescent dye, the hydrophobicity of the solvent and/or of the further solvent.

Furthermore, the release rate of the fluorescent dye from the solution and/or the set solution may be controlled by the relative log P values of the different components of the solution, as described herein above in the section ‘Physics of the solution’. As an example a solution comprising water insoluble carbohydrates and solvents which are fully compatible, i.e. having similar log P, will result in higher release of the fluorescent dye from the solution and/or the set solution. In cases wherein, the carbohydrate and solvent are fully compatible, i.e. have similar log P as e.g. LOIB and GTO or SAIB and GTH, one phase systems are predominantly formed, which are unable to retain fluorescent dyes based on the kinetic trap principle due to the reduced viscosity caused by the presence of the co-solvent.

In cases wherein a carbohydrate is mixed with a co-solvent to form fiducial markers with reduced viscosities, controlled release of entrapped compounds may be facilitated based on diffusion limited kinetics. Entrapped compounds, say a NIRdye-polymer construct may be released in a controlled manner defined by the viscosity of the depot and or the molecular cross-section of the diffusing NIRdye-polymer construct. Such polymer construct may comprise PNIPAM, cellulose acetate butyrate, cellulose acetate, perfluorocarbons, poloxamer pluronics, polyethylene glycol (PEG), polylactic acid (PLA), poly(lactic-co-glycolic acid) (PLGA), poly(L-lactide) (PLA), poly(glycolide) (PGA), ploy(DL-lactide) (DLPLA), poly(dioxanone) (PDO), poly(DL-lactide-co-L-lactide) (LDLPLA), poly(DL-lactide-co-glycolide) (DLPLG), poly(glycolide-co-trimethylene carbonate) PGA-TMC, poly(L-lactide-co-glycolide) (LPLG) or poly(caprolactone) (PCL) conjugated to a fluorescent dye such as of phthalocyanine, naphthalocyanines, porphines, antracocyanine. The release rate from the depot is controlled by the size (molecular cross-section), the entanglement and interactions of the polymer with the solution, and the solubility of the polymer in aqueous media.

In other embodiment of the disclosure, fluorescent particles or rods such as quantum dots or carbon nanotubes may be released from the set solution. Yet in another embodiment, polymer or metallic nanoparticles entrapping or surface functionalized with fluorescent dyes may be released utilizing the similar principles for achieving controlled release.

Upon release of fluorescently labelled particles, polymers or rods, these spread by diffusion in the tumor tissue and accumulate in the draining lymph nodes. The degree of lymph node accumulation depends on polymer size, hydrophobicity and or conjugation of targeting ligands.

In some embodiments of the disclosure, the released constructs may carry diagnostic isotopes for SPECT/PET or gamma-probe detection of the draining lymph nodes. In another embodiment of the disclosure, the released constructs may include a pro-drug for treatment e.g. of metastatic cancer in the draining lymph nodes. In another embodiment of the disclosure, the released constructs may include an enzyme, redox or pH activatable fluorescent dye for functional imaging of the tumor or lymph nodes.

In one embodiment, the fluorescent dye is covalently conjugated to polyethylene glycol (PEG) and has a molecular weight above 2000 Da. Conjugation of the fluorescent dye to a hydrophilic PEG polymer may provide release of the fluorescent dye from the solution under aqueous conditions.

In one embodiment, the released fluorescent dye-PEG conjugate accumulates in lymph nodes following release from the solution in vivo.

In one embodiment, the fluorescent dye-PEG conjugate has a molecular weight above 2000 Da, such as above 3000 Da, for example above 4000 Da, such as above 5000 Da, for example above 10000 Da such as above 15000 Da, for example above 20000 Da.

Use of the Solution

The solution according to the present disclosure, may be liquids with viscosities in the range 100-1000 cP, which enable percutaneous, endoscopic or bronchoscopic administration though thin injection needles into almost any site in the human body.

Thus, one aspect of the present disclosure relates to use of the solution as described herein as a fiducial marker.

Guidance for Surgery

As described above, there is a need for in the field for development of good fiducial markers to use with the developing technologies in the field of image-guided surgery.

The solution as described herein may comprise multimodal image modalities relevant for surgical guidance as fiducial markers. These solutions comprise water insoluble carbohydrates that inherently have negative contrast on MRI due to their negligible water content, and ultrasound contrast due to their inherent high viscosity and ductility compared to soft tissues. These solutions may further enable CT imaging via incorporation of iodinates carbohydate esters, iodinated polymers or gold nanoparticles and SPECT/PET imaging via coordination of diagnostic radionuclides to the fluorescent dye entrapped in the solution.

During injection of the solution, the position and volume change of the fiducial marker can be monitored in real-time either via ultrasound imaging or x-ray-based technologies e.g. fluoroscopy for solutions including radiopaque constituents such as xSAIB. The healthcare professional injecting the marker is thus able to evaluate the quality and precision of fiducial marker before advancing with the surgical procedure. When in doubt about the injection quality, x-ray-based imaging, e.g. CT, may further be employed to ensure correct position of the marker before proceeding with the surgical intervention. Such multimodal markers have high potential in image guided surgery, which will improve surgical and therapeutic intervention, reduce discomfort and post-operative pain for the patient, improve survival, shorten hospitalization, and lower healthcare costs.

The solution of the present disclosure may be used for all surgical and interventional procedures/indications, where fiducial markers are warranted for guidance.

Thus, in one embodiment, the present disclosure relates to use of the solution as described herein for guidance of surgery.

In one embodiment, the solution as described herein is used for labelling of a reference point following a surgical procedure. As an example, the solution as described herein may be used for labelling of a reference point of a tissue biopsy procedure or positioning of a therapeutic device.

Guidance for External Beam Radiation Therapy

Radiotherapy is a cost-effective and widely-adopted solution to cancer therapy, with over 50% of patients diagnosed with solid tumors undergoing some form of radiation treatment. In the US and Europe alone more than 2.5 million patients will receive radiotherapy on an annual basis. The prevalence of this treatment option is also reflected by the fact that over 1000 medical centers in Europe are equipped with radiotherapy equipment, known as linear accelerators or linacs. The radiotherapy treatment is most often delivered over several treatment fractions—sometimes up to 30—and the ultimate key to an effective treatment is to hit the tumor precisely on each of these treatment fractions. For some cancers their location and soft tissue contrast is too similar to surrounding tissues for accurate delination based on the x-ray-based imaging technologies included in the linear accelerator equipment. For such cases the inclusion of fiducial markers with high radiocontrast can provide reference points for accurate positioning of patients and delivery of radiation therapy.

To be able to achieve this, soft tissue markers, or fiducial markers are needed.

The technological advancements in radiotherapy have enabled the rise of new and optimized treatment approaches, all of which relies on improved image-guided treatment and the need for soft tissue markers:

Thus, in one aspect, the present disclosure relates to a solution as described herein for guidance of EBRT.

In one embodiment, the present disclosure relates to use of the solution as described herein for guidance of external beam radiation therapy.

Labelling of Sites with Specific Enzymatic Activity/Environment (w Cleavable Quencher)

In one embodiment, the fluorescent dye of the solution of the present disclosure is conjugated to a quencher.

The fluorescent dye-quencher conjugate may be cleaved under conditions, such as by specific enzymatic reactions, by low pH or by a change in redox potential.

Thus, in the fluorescent dye-quencher conjugate, no signal is provided from the solution. However, upon controlled release of the fluorescent dye-quencher conjugate from the set solution, the fluorescent dye may be activated at sites of interest having e.g. a low pH, a specific redox potential or the presence of a given enzyme.

Combination Treatment

The solution of the present disclosure offers a range of possibilities for combination treatments, either by utilizing the current disclosure in combination with other therapies, or by including extra/other features on top of the fiducial marker function already described.

The solution of the present disclosure may be co-formulated with different active pharmaceutical ingredients, such as wound healing or disinfection agents. Upon injection of such solutions, wound healing or disinfection agents are released locally. Alternatively, the solution of the present disclosure may be co-formulated with antibiotic agents.

In yet another embodiment of the disclosure, photo-sensitisers for photo dynamic therapy (PDT), such as phthalocyanine or naphthalocyanine derivatives with reduced affinity for the solution (reduced log P), may be released for optimal accumulation of the photo-sensitizer in the tumor tissue and allow for following activation of the photo-sensitizer by light leading to the destruction of cancer cells.

Kit for Preparation

The present disclosure may further relate to a kit for preparation of the solution of the disclosure nearby or at the site of administration. This may be advantageous in cases where the fluorescent dye is to be coordinated with a radionuclide since the non-radioactive components of the solution may be provided and stored at e.g. the hospital, whereas the radioactive source may be provided at a needs basis and applied ultimately after being received or generated at site.

Thus, in one embodiment, the present disclosure relates to a kit comprising
a. a solution comprising a water insoluble carbohydrate, a solvent having a log P in the range of −2 to 2, and optionally a further solvent and/or a further imaging agent, as described herein,
b. a solution comprising a fluorescent dye as described herein.

In one embodiment, the kit further comprises a radionuclide as part of component b) or as an individual component c).

In one embodiment, the components of component a) of the kit are provided as separate parts, or as two components comprising a) solid components and b) liquid components.

In one embodiment, the components of component b) of the kit are provided as separate parts, or as two components comprising a) solid components and b) liquid components.

Items
1. A solution comprising
   a. a water insoluble carbohydrate,
   b. a fluorescent dye, and
   c. an organic solvent having a log P in the range of −2 to 2.
2. The solution according to item 1 wherein the fluorescent dye has a log P above 2, thereby providing retention of the fluorescent dye in the solution under aqueous conditions.
3. The solution according to any one of the preceding items, wherein the fluorescent dye has a log P above 2, such as above 3, for example above 4, such as above 5, for example above 6, such as above 8, for example above 10, such as above 15.
4. The solution according to any one of the preceding items, wherein less than 10% of the fluorescent dye is released after 5h under aqueous conditions, such as less than 5%.
5. The solution according to item 1 wherein the fluorescent dye is covalently conjugated to polyethylene glycol and has a molecular weight above 2000 Da, thereby providing release of the fluorescent dye from the solution under aqueous conditions.
6. The solution according to item 5, wherein the released fluorescent dye-PEG conjugate accumulates in lymph nodes following release from the solution in vivo.
7. The solution according to items 5 and 6, wherein the fluorescent dye-PEG conjugate has a molecular weight above 2000 Da, such as above 3000 Da, for example above 4000 Da, such as above 5000 Da, for example above 10000 Da such as above 15000 Da, for example above 20000 Da.
8. The solution according to any one of the preceding items, wherein the fluorescent dye is part of a particle.
9. The solution according to item 8, wherein the particle is selected from the group consisting of quantum dots, metallic nanoparticles functionalized with a fluorescent dye, polymeric nanoparticles functionalized with a fluorescent dye and carbon nanotubes.
10. The solution according to any one of items 8 to 9, wherein the particle is coated with a polymer.
11. The solution according to any one of the preceding items, wherein the fluorescent dye is a near infrared (NIR) contrast agent.
12. The solution according to any one of the preceding items, wherein the fluorescent dye is a NIR-I contrast agent.
13. The solution according to any one of the preceding items, wherein the fluorescent dye is a NIR-II contrast agent.
14. The solution according to any one of the preceding items, wherein NIR contrast agent is selected from the group consisting of phthalocyanines, naphthalocyanines, porphines, antracocyanine and cyanine dyes.
15. The solution according to any one of the preceding items, wherein NIR contrast agent is selected from the group consisting of phthalocyanine, naphthalocyanines, porphines, antracocyanine.
16. The solution according to any one of the preceding items, wherein NIR contrast agent is a phthalocyanine, such as PC1, PC2 and/or PC3.
17. The solution according to any one of the preceding items, wherein the fluorescent dye is conjugated to a polymer.
18. The solution according to any one of the preceding items, wherein the fluorescent dye emits photons in the sub-NIR range.
19. The solution according to any one of the preceding items, wherein the fluorescent dye emits photons in the 700-900 nm range.
20. The solution according to any one of the preceding items, wherein the fluorescent dye emits photons in the range above 900 nm.
21. The solution according to any one of the preceding items, wherein the water insoluble carbohydrate comprises one or more hydroxyl groups functionalized to form C2-C7 esters.
22. The solution according to any one of the preceding items, wherein the C2-C7 esters are formed by a bond between the hydroxyl group(s) of the carbohydrate and the carbonyl group of a C2-C7 alkanoyl(s).
23. The solution according to any one of the preceding items, wherein the number of hydroxyl groups functionalized to form C2-C7 esters is n, n-1, n-2, n-3, n-4 or n-5, wherein n is the total number of hydroxyl groups of the carbohydrate.
24. The solution according to any one of the preceding items, wherein all hydroxyl groups are functionalized to form C2-C7 esters.
25. The solution according to any one of the preceding items, wherein the water insoluble carbohydrate is selected from the group consisting of monosaccharides, disaccharides, trisaccharides and oligosaccharides.
26. The solution according to any one of the preceding items, wherein the water insoluble carbohydrate is a monosaccharide selected from the group consisting of Glucosamine, Galactosamine, Mannosamine, Mannose, Rhamnose, Rhamnosamine, Galactose, Allose, Allosamine, Altrose, Altrosamine, Gulose, Gulosamine, Idose, Idosamine, Talose and Talosamine.
27. The solution according to any one of the preceding items, wherein the water insoluble carbohydrate is a disaccharide selected from the group consisting of maltose, trehalose, lactose, sucrose, Galp-(1→2)-Glc, Galp-(1→3)-GlcN, Galp-(1→4)-Glc, Glcp-(1→4)-Glc, Glcp-(1→6)-Glc, Glcp-(1→2)-GlcN, Galp-(1→4)-ManN, Glcp-(1→4)-GalN, Manp-(1→3)-Glc, ManNp-(1→4)-Gal, GalNp-(1→3)-ManN, GlcNp-(1→6)-GalN, Rhamnp-(1→6)-Glc, Glcp-(1→1)-Glcp, Talp-(1→4)-Glu, Glup (1→3)-Ido, GlcNp-(1→4)-GlcN, GlcNp-(1→6)-GlcN.
28. The solution according to any one of the preceding items, wherein the water insoluble carbohydrate is a trisaccharide selected from the group consisting of raffinose, Galp-(1→2)-Glcp-(1→3)-Galp, Galp-(1→4)-Glcp-(1→6)-GlcN, Galp-(1→4)-Glcp-(1→6)-Gal, Glcp-(1→4)-Glcp-(1→4)-Glcp, Glcp-(1→6)-Glcp-(1→6)-Glc, Galp-(1→6)-Glcp (1→2)-Fruf, Glcp-(1→3)-Fruf-(2→1)-Glcp, Galp-(1→4)-ManNp-(1→3)-Glu, Glcp-(1→4)-GalN-(1→2)-Man, Manp-(1→3)-Glcp-(1→4)-GlcN, ManNp-(1→4)-Galp-(1→3)-Glc, GalNp-(1→3)-ManNp-(1→6)-GlcN. Rhamnp-(1→6)-Glcp-(1→4)-GlcN, Galp-(1→6)-Glcp-(1→1)-Glcp, Talp-(1→4)-Glup-(1→2)-Man, Glup (1→3)-Idop-(1→6)-Glu, GlcNp-(1→6)-GlcNp (1→4)-GlcN.
29. The solution according to any one of the preceding items, wherein the water insoluble carbohydrate is a oligosaccharide selected from the group consisting of Galp-(1→4)-Glcp-(1→6)-glcp-(1→4)-Glc, Galp-(1→4)-Glcp-(1→4)-Glcp-(1→4)-Glcp-(1→4)-Glc, Galp-(1→4)-Glcp-(1→4)-Galp-(1→4)-Glc, Glcp-(1→4)-Glcp-(1→4)-Glcp-(1→4)-Glc, Galp-(1→6)-Glcp-(1→6)-Galp-(1→6)-Glc, Galp-(1→6)-Glcp-(1→6)-Galp-(1→4)-Glc, Galp-(1→6)-Glcp-(1→6)-Glcp-(1→4)-Glc, GlcNp-(1→4)-GlcNp-(1→6)-GlcNp-(1→4)-GlcN, GlcNp-(1→6)-Galp-(1→6)-Glcp-(1→2)-Fruf, Galp-(1→4)-Glcp-(1→3)-Fruf-(2→1)-Glcp, Talp-(1→4)-Glup-(1→2)-Man-(1-3)-Glu, Glup (1→3)-Idop-(1→6)-Glup-(1→2)-Gal, cellulose acetate, cellulose acetate butyrate and cellulose acetate proprionate.
30. The solution according to any one of the preceding items, wherein the C2-C7 alkanoyl is selected from acetyl, propanoyl, butanoyl, isobutanoyl, pentanoyl, hexanoyl, heptanoyl and benzoyl.
31. The solution according to any one of the preceding items, wherein the C2-C7 alkanoyl is selected from acetyl, propanoyl, isobutanoyl and benzoyl.
32. The solution according to any one of the preceding items, wherein the water insoluble carbohydrate has a structure according to formula (I),

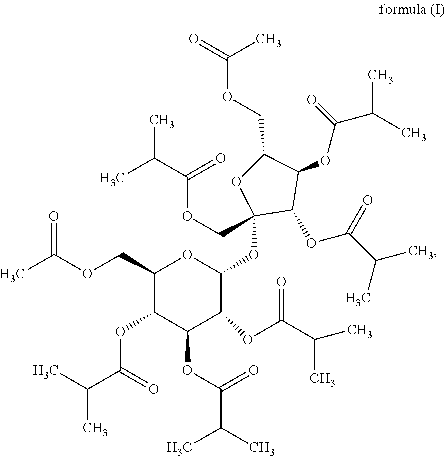 [SMILES: CC(=O)OC[C@H]1O[C@@](COC(=O)C(C)C)(O[C@H]2O[C@H](COC(C)=O)[C@@H](OC(=O)C(C)C)[C@H](OC(=O)C(C)C)[C@H]2OC(=O)C(C)C)[C@@H](OC(=O)C(C)C)[C@@H]1OC(=O)C(C)C]

33. The solution according to any one of the preceding items, wherein the water insoluble carbohydrate has a structure according to formula (II),

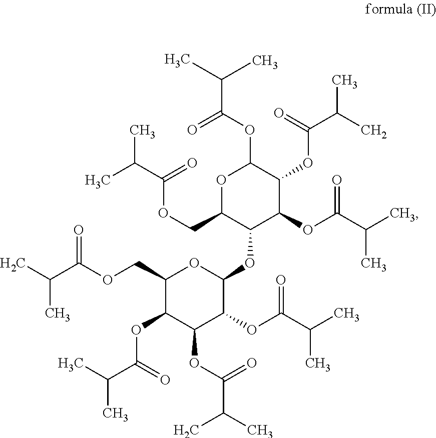

34. The solution according to any one of the preceding items, wherein the organic solvent diffuses out of the solution under aqueous conditions, providing a gel, a glass, a semi-solid, a solid, a crystal or any combination thereof.
35. The solution according to any one of the preceding items, wherein the viscosity increases by more than 1000 centipose (cP) under aqueous conditions, such as more than 5000 cP, for example more than 10000, such as more than 50000 cP, for example more than 100000 cP.
36. The solution according to any one of the preceding items, wherein the solution sets under aqueous conditions in less than 5 h, such as less than 4 h, for example less than 3 h, such as less than 2 h.
37. The solution according to any one of the preceding items, wherein the aqueous conditions are in vitro conditions, such as a buffer system.
38. The solution according to any one of the preceding items, wherein the aqueous conditions are in vivo conditions.
39. The solution according to any one of the preceding items, wherein the organic solvent has a log P in the range of −2 to 2, for example in the range of −1.8 to 1.8, such as in the range of −1.5 to 1.5, for example in the range of −1 to 1, such as in the range of −2 to 1, for example in the range of −1.5 to 1, for example in the range of −1 to 2, such as in the range of −1 to 1.5.
40. The solution according to any one of the preceding items, wherein the organic solvent is an alcohol.
41. The solution according to any one of the preceding items, wherein the organic solvent is a C1-C4 alcohol.
42. The solution according to any one of the preceding items, wherein the organic solvent is selected from the list consisting of methanol, ethanol, propanol, isopropanol, butanol, isobutanol, tert-butanol, benzyl alcohol, propylene carbonate and dimethyl sulfoxide.
43. The solution according to any one of the preceding items, wherein the amount of organic solvent is in the range of 1 to 30%, for example 1 to 20%, such as 1 to 15%, for example 1 to 10%, such as 5 to 10%.
44. The solution according to any one of the preceding items, further comprising a monoglyceride, diglyceride and/or triglyceride.
45. The solution according to any one of the preceding items, wherein the triglyceride is selected from the group consisting of glyceryl tridecanoate (GTD), glyceryl trioctanoate (GTO) and glyceryl trihexanoate (GTH).
46. The solution according to any one of the preceding items, wherein the amount of monoglyceride, diglyceride and/or triglyceride is in the range of 0 to 50%, such as in the range of 0 to 40%, for example in the range of 0 to 30%, such as in the range of 0 to 20%, for example in the range of 0 to 10%.
47. The solution according to any one of the preceding items, wherein the fluorescent dye is coordinated to a radionuclide.
48. The solution according to any one of the preceding items, wherein the radionuclide is selected from the group consisting of Tc-99m, In-111, Ga-67, Lu-177, Tl-201, Sn-117m, Cu-64, Mn-52, Zr-89, Co-55, Sc-44, Ti-45, Sc-43, Cu-61, As-72, Te-152, F-18, Ga-68, C-11, Nd-140 and Te-149.
49. The solution according to any one of the preceding items, comprising Cu-64 and PC1, PC2, and/or PC3.
50. The solution according to any one of the preceding items, comprising a further imaging agent.
51. The solution according to any one of the preceding items, wherein the further imaging agent is selected from the group consisting of X-ray agent, CT agent, MRI agent, PET agent and SPECT agent.
52. The solution according to any one of the preceding items, wherein the further imaging agent has a structure according to formula (III),

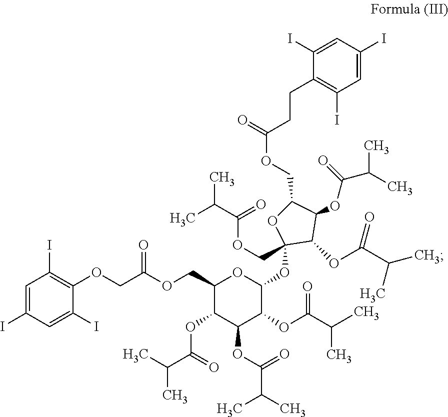 [SMILES: CC(C)C(=O)OC[C@@]1(O[C@H]2O[C@H](COC(=O)COc3c(I)cc(I)cc3I)[C@@H](OC(=O)C(C)C)[C@H](OC(=O)C(C)C)[C@H]2OC(=O)C(C)C)O[C@H](COC(=O)CCc2c(I)cc(I)cc2I)[C@@H](OC(=O)C(C)C)[C@@H]1OC(=O)C(C)C]

53. A solution according to any one of the preceding items for use as an in vivo diagnostics tool.
54. A method of in vivo imaging, the method comprising
   a. administering a solution according to any one of the preceding items to an individual in need thereof,
   b. excitation of the fluorescent dye, and
   c. detection of the fluorescent dye.
55. The method according to any one of the preceding items, wherein the solution is administered by injection and/or smearing.
56. Use of the solution according to any one of the preceding items for in vivo imaging.
57. Use of the solution according to any one of the preceding items as a fiducial marker.
58. Use of the solution according to any one of the preceding items for guidance of surgery.

### EXAMPLES

### Example 1. Hydrophobicity and Molecular Weight of Fluorescent Dyes and Gel Materials

Conventional and near infrared (NIR) fluorophores are often comprised of highly conjugated molecules characterized of being hydrophobic (log P>0) unless chemically modified to be soluble in aqueous solution by incorporation of charged residues and or hydrophilic polymers such as PEG. The hydrophobic characteristics of such dyes ensured good compatibility with the hydrophobic solutions and enable high solution retention. Examples of such dyes are described in table 1 whereas relevant solution materials are described in table 2

Log P values were obtained by calculations based on the algorithm of Viswanadhan et al (Viswanadhan, V. N.; Ghose, A. K.; Revankar, G. R.; Robins, R. K., J. Chem. Inf. Comput. Sci., 1989, 29, 163-172). The log P value can also be determined by octanol-water partitioning experiment. Positive log P values are characteristic hydrophobic compounds, whereas negative log P values indicate a hydrophilic compound.

TABLE 1
LogP, molecular weight and maximum absorption wavelength for selected dyes

| | | | UVvis |
|---|---|---|---|
| | | Mw | maximum |
| Dyes | LogP | (g/mol) | (nm) |
| Porphine | | | |
| 2,3,7,8,12,13,17,18-Octaethyl-21H,23H-porphine | 12.3 | 534.8 | 401 |
| 5,10,15,20-Tetraphenyl-21H,23H-porphine | 11.22 | 614.7 | 514 |
| Phthalocyanine | 6.49 | 514.5 | 698 |
| 2,9,16,23-Tetra-tert-butyl-29H,31H-phthalocyanine | 10.04 | 736.97 | 696 |
| 1,4,8,11,15,18,22,25-Octabutoxy-29H,31 H- | 15.82 | 1091.4 | 762 |
| phthalocyanine | | | |
| 2,3,9,10,16,17,23,24-Octakis(octyloxy)-29H,31H- | 30.05 | 1540.3 | 701 |
| phthalocyanine | | | |
| Naphthalocyanine | | | |
| 2,11,20,29-Tetra-tert-butyl-2,3-naphthalocyanine | 10.45 | 714.2 | 712 |
| 5,9,14,18,23,27,32,36-Octabutoxy-2,3- | 16.63 | 939.2 | 784 |
| naphthalocyanine | 19.65 | 1291.6 | 867 |
| Antracocyanine | 14.4 | 915.03 | — |
| Cyanine dyes | | | |
| Cy5-alkyne | 4.09 | 520.7 | 646 |
| Cy5.5-alkyne | 6.07 | 620.9 | 684 |
| Cy7-alkyne | 5.09 | 586.8 | 750 |
| Cy7.5-alkyne | 7.07 | 686.9 | 788 |

TABLE 2
LogP and molecular weight of selected solution matrix compounds.

| Carbohydrate esters | LogP | Mw (g/mol) |
|---|---|---|
| Glucose pentapropionate | 2.78 | 460.5 |
| Glucose pentaisobutyrate | 5.49 | 530.6 |
| Glucose pentabenzoate | 9.54 | 700.7 |
| Lactose octapropionate (LOP) | 4.43 | 790.80 |
| Sucrose acetate isobutyrate (SAIB) | 6.46 | 846.40 |
| Lactose octaisobutyrate (LOIB) | 8.77 | 903.03 |
| Sucrose octabenzoate (SOB) | 15.30 | 1145.14 |
| Solvents | | |
| Dimethylsulfoxid (DMSO) | −1.40 | 78.13 |
| Ethanol (EtOH) | −0.16 | 46.07 |
| Propylenecarbonate (PC) | 0.79 | 102.09 |
| Benzyl alcohol (BA) | 1.21 | 108.14 |
| Co-solvents | | |
| Glycerol trihexanoate (GTH) | 5.59 | 386.50 |
| Glyceryl trioctanoate (GTO) | 8.25 | 470.68 |
| Glyceryl tridecanoate (GTD) | 10.92 | 554.80 |

### Discussion

The selected dyes are exited in the optical range from visible light (400-600 nm) and up into the NIR-I range (700-900 nm). The dyes in table 1 are all hydrophobic with log P values larger than 4 and display a positive linear correlation between molecular weight and log P (R²=0.86). The dyes with the larges molecular weights are thus predicted to be retained more effectively in the solutions described in this disclosure due to both enhanced affinity via hydrophobic interactions with the solution as well as impaired diffusion in the viscous solution caused by a larger molecular cross section.

The fluorophores capable of chelating cations such as radionuclides are represented by porphines, phthalocyanines, naphthalocyanines and antracocyanine in table 1. All are larger constructs with high log P values indicating high solution retention compared to the smaller cyanine dyes Cy5-Cy7.5. The water solubility of such dyes is very low and minimal release of such dyes is expected.

The hydrophobicity (log P) of the carbohydrate esters and triglyceride co-solvents were found to increase with increasing acyl-chain length. Solvents applicable for gel formation were found to span a range of log P values from −1.4 to 1.21 indicating their propensity for diffusing out of the solution upon injection into aqueous media and causing non-solvent induced phase separation. Triglyceride co-solvents were found to span the log P range from 5.59 to 10.92, whereas the carbohydrate esters span the log P range from −1.17 to 15.30.

Specific matches in log P values were identified between co-solvents and carbohydrate esters, e.g the co-solvent carbohydrate mixtures SAIB:GTH and LOIB:GTO have differences in log P value of less than one indicating similar hydrophobicity and compatibility. Large differences in log P value between co-solvent and the carbohydrate ester may be a predictor of incompatibility leading to micro phase separation of the solution, see further in section ‘Physics of the solution’.

Conclusion

Log P values have been established by computational methods for selected solution matrix and dye compounds, and have been utilized to predict compatibility between co-solvents and carbohydrate esters, as well as predicting higher retention of the chelating dyes.

### Example 2. Materials and Methods

All chemicals were purchased from Sigma-Aldrich unless otherwise stated. Reagents for ICP-MS measurements were TraceSelect® and nitric acid was purchased from Fluka Analytical. Premixed lipid mixture, composed of Hydrogenated Soy L-α-phosphatidylcholine (HSPC), Cholesterol (CHOL) and 1,2-distearoyl-sn-glycero-3-phosphoethanolamine-N-[methoxy (polyethy-lene glycol)-2000] (ammonium salt) (DSPE-PEG2000) in the molar ratio of HSPC:CHOL:DSPE-PEG2000 (56.5:38.2:5.3), was purchased from Lipoid GmbH.

Sucrose acetate isobutyrate (SAIB) was purchased from Sigma and Lactose octaisobutyrate (LOIB) and xSAIB were prepared by inhouse custom synthesis.

125I ([¹²⁵I]NaI) and the LCS cocktail, Ultima gold, was purchased from Perkin Elmer.

CT26 (murine colon carcinoma) was purchased from ATTC (Rockville, Md., USA). DMEM medium supplemented with 10% fetal calf serum and pen-strep was purchased from Invitrogen Inc. (Denmark).

All water used was collected from a Milli-Q system (Millipore). TRIS iso-osmotic buffer (10 mM TRIS, 150 mM NaCl) was prepared in Milli-Q water and adjusted to pH 7.8 with HCl.

Abbreviations:

8HQ: 8-hydroxyquinoline

SAIB: sucrose diacetate hexaisobutyrate, Formula (I)

LOIB: lactose octaisobutyrate, Formula (II)

xSAIB: iodinated SAIB-derivative according to formula (III)

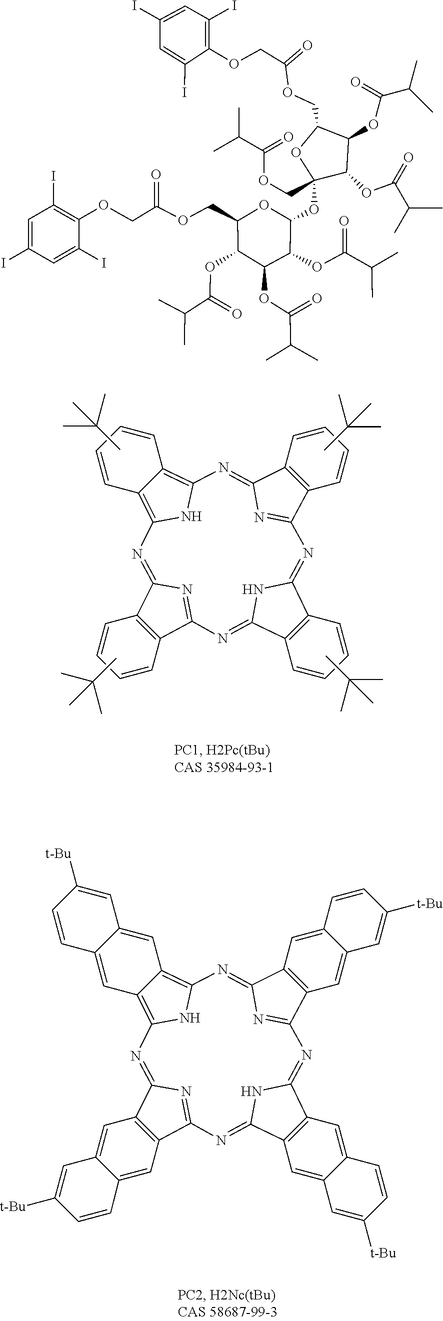

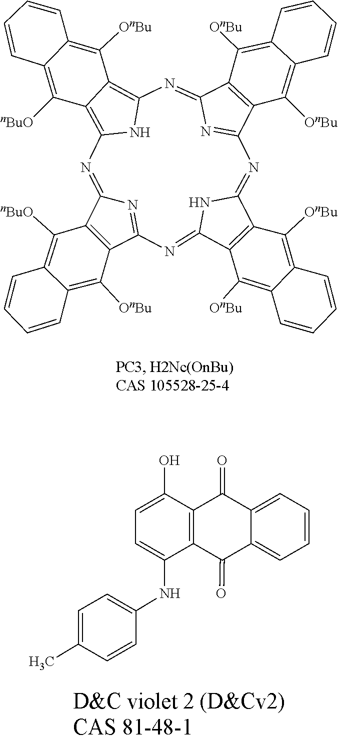

### Example 3. Synthesis of 6′-(Cyanine 7.5)-Isobutyric Sucrose (Sucrose Septaisobutyrate Cyanine 7.5, SSIB-Cy7.5)

In the current example, the NIR dye Cyanine 7.5 is chemically linked to the hydrophobic carbohydrate ester sucrose septaisobutyrate yielding the product SSIB-Cy7.5.

Methods:

All reactions were carried out under inert atmosphere (N₂). Water sensitive liquids and solutions were transferred via syringe. Water used for washing of the isolated products was in all cases MilliQ water. Organic solutions were concentrated by rotary evaporation at 30-60° C. at 200-0 mbar. Thin layer chromatography (TLC) was carried out using aluminum sheets pre-coated with silica 60F (Merck 5554). The TLC plates were inspected under UV light or developed using a cerium ammonium sulphate solution (1% cerium (IV) sulphate and 2.5% hexa-ammonium molybdate in a 10% sulfuric acid solution).

Reagents: Cyanine 7.5 NHS ester was purchased from Lumiprobe, and dry solvents were purchased from Acros Organics (AcroSeal, extra dry over molecular sieves). All other chemicals were purchased from Sigma Aldrich and were used as received. Instrumentation: Nuclear Magnetic Resonance (NMR) of intermediates was acquired on a Bruker Ascend™ 400 MHz—operating at 401.3 MHz for ¹H and 100.62 MHz for ¹³C—with a 5 mm H—Broadband Dual Channel z-gradient Prodigy cryoprobe at 298 K, using the residual non-deutorated solvent residue in the nmr solvents as internal standard. NMR of the final product was acquired with an 800 MHz Bruker Avance IIIHD spectrometer equipped with a TCI cryoprobe (Bruker) in order to obtain optimal spectral resolution. All coupling constants (J) are expressed in Hz. The FID files were processed in Mnova Suite. MALDI-TOF MS was acquired on a Bruker Autoflex Speed™ mass spectrometer. The matrix used for MALDI-TOF was a mixture of 2,5 dihydroxy benzoic acid (DHB) spiked with sodium trifluoroacetate in ethanol (60 mg/mL). UPLC was conducted on a Waters Acquity Ultra performance LC system with Binary solvent manager and TUV detector. Preparative HPLC was conducted on a Waters 600 pump and controller with a Waters 2489 UV/Vis detector.

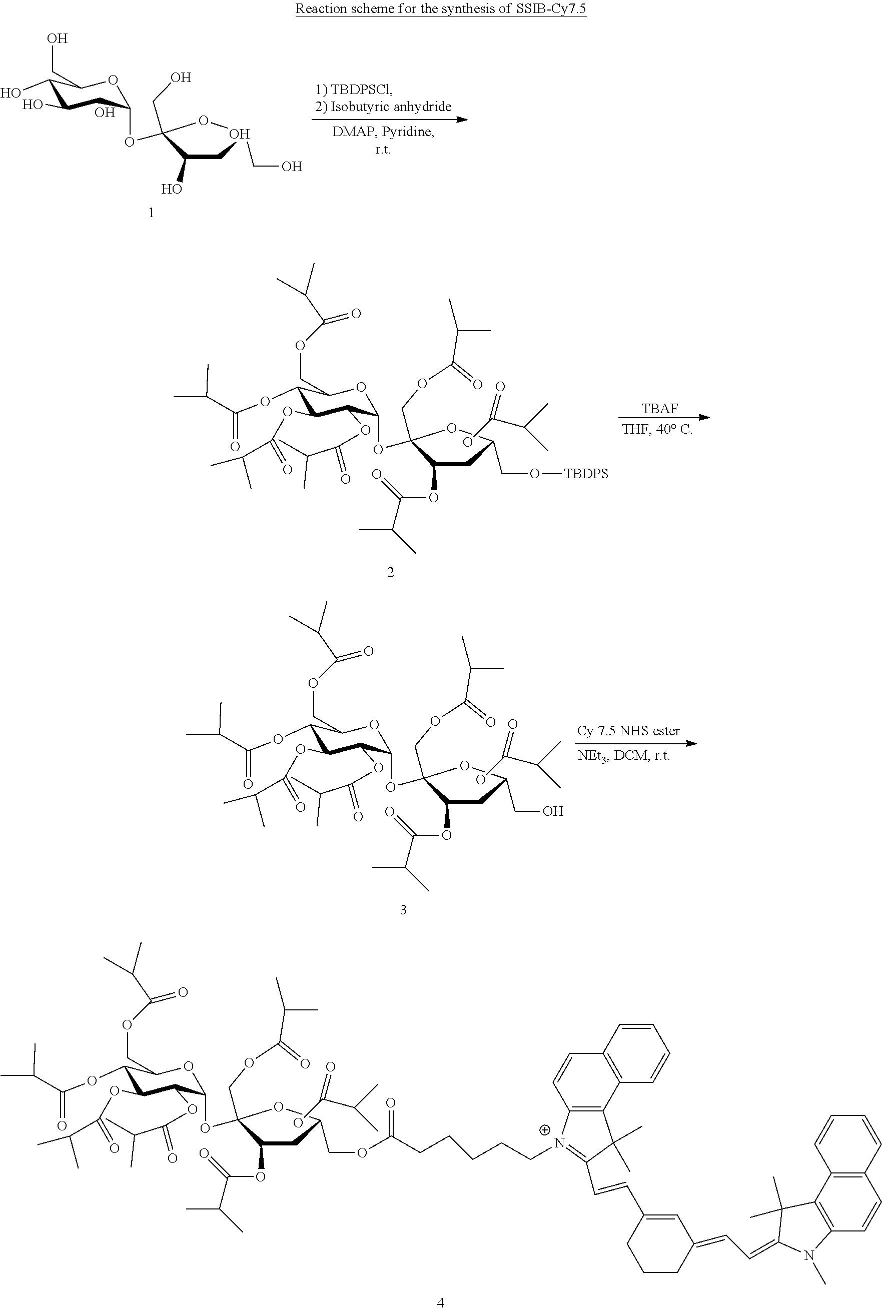

6′-TBDPS-Isobutyric Sucrose (2)

Sucrose (1) (2.5 g, 7.3 mmol) was suspended in a solvent mix of 35 mL dry pyridine and 10 mL dry DMF. Hereafter, DMAP (0.36 g, 2.92 mmol) was added and the mixture was stirred until properly dissolved. Then, TBDPS-Cl (1.1 mL, 4.0 mmol (0.55 eq)) was added dropwise through a syringe over 10-15 minutes, and the reaction was continued overnight. 16 hours later, another portion of TBDPS-Cl (1.1 mL, 4.0 mmol (0.55 eq)) was added, after which the reaction was again left to stir overnight. Hereafter, UPLC (column: C8. Injection volume: 5 μL. Eluent: A: 0.1% formic acid in water. B: Acetonitrile, 0.1% formic acid. Gradient: 5-100% B within 6 minutes. Wavelengths 220 and 280 nm) showed conversion to mono and di-tbdps sucrose in a 2:1 relationship (retention times: 3.1 and 5.2 minutes respectively). The formed 6′-TBDPS-sucrose was not isolated, instead the mixture was reacted directly with isobutyric anhydride and then purified to give (2). Isobutyric anhydride (34 mL, 0.21 mol) was added, and the reaction was stirred at room temperature for 1 day. The reaction was followed by MALDI-TOF MS. At the point of completion, the reaction mixture was concentrated on celite in vacuo. Purification was done by flash chromatography (EtOAc in hexane with 2% increments). Yield=14.8 g (50%). ¹H-NMR (400 MHz, DMSO-D6): ¹H NMR (400 MHz, DMSO-d₆) δ 7.63-7.57 (m, 4H), 7.47-7.37 (m, 6H), 5.62 (d, J=3.6 Hz, 1H), 5.51-5.46 (m, 2H), 5.39-5.33 (m, 1H), 5.04 (t, J=9.8 Hz, 1H), 4.87 (dd, J=10.4, 3.7 Hz, 1H), 4.25 (ddd, J=10.3, 4.2, 2.0 Hz, 1H), 4.20-4.03 (m, 4H), 3.91 (dd, J=12.5, 2.0 Hz, 1H), 3.87-3.77 (m, 2H), 2.60-2.51 (m, 3H), 2.47-2.33 (m, 3H), 1.15-1.07 (m, 12H), 1.07-0.94 (m, 40H).

¹³C-NMR (101 MHz, DMSO-d₆): δ 175.6, 175.2 (2C), 175.0, 174.9 (2C), 174.5, 135.0 (4 C), 132.4, 132.2, 129.9 (2C), 127.8 (4C), 102.4, 89.1, 80.0, 75.2, 73.5, 69.2 69.1, 68.1, 67.1, 63.8, 63.3, 61.2 (carbohydrate carbons), 33.2 (3C), 33.1 (2C), 33.0 (2C) (CH isobutyrate), 26.4 (4C), 18.7, 18.6 (3C), 18.5 (5C), 18.4 (2C), 18.3 (2C), 18.2.

MALDI-TOF MS: Calc [M+Na]: 1093.53. Found:1093.31.

6′-OH-Isobutyric Sucrose (Sucrose Septaisobutyrate) (3)

6′-TBDPS-isobutyric sucrose (2) (14.8 g, 13.8 mmol) was dissolved in dry THF (80 mL). Acetic acid (12 mL, 0.21 mol) was carefully added dropwise. The reaction mixture was then cooled down, and 1.0M TBAF solution in THF (83 mL, 83 mmol) was added over 10-15 minutes through a syringe. The reaction was allowed to heat to room temperature over 30 minutes, hereafter it was warmed to 40° C. and stirred at this temperature overnight. Then, TLC (Hexane:Ethyl acetate 3:1) showed completion of the reaction (rf product: 0.2). The reaction mixture was cooled to room temperature and first hexane (300 mL) then demineralized water (300 mL) was added. The mixture was stirred for 10 minutes, and hereafter poured into a separatory funnel. The organic phase was collected and the water phase was extracted with hexane (2×300 mL). The combined organic phases were washed with HCl (aq) (500 mL, pH=2) and subsequently with phosphate buffer (3×300 mL, pH=6.8). The organic phase was concentrated on celite and then purified by dry column purification (EtOAc in hexane with 2-4% increments) to give the product. Yield 5.1 g (89.5%). Texture: transparent oil. ¹H-NMR (400 MHz, Chloroform-d): δ 5.68-5.39 (m, 4H), 5.18 (t, J=10.4 Hz, 1H), 4.94 (dd, J=10.4, 3.6 Hz, 1H), 4.36-4.14 (m, 3H), 4.10-3.93 (m, 3H), 3.84 (dd, J=12.9, 2.8 Hz, 1H), 3.60 (dd, J=12.9, 3.6 Hz, 1H), 2.69-2.24 (m, 7H), 1.34-0.99 (m, 42H).

¹³C-NMR (101 MHz, Chloroform-d): δ 176.8, 176.3 (2C), 176.1, 176.0, 175.9, 175.2, 102.8, 90.2, 81.4, 75.6, 72.5, 70.0, 69.5, 69.1, 67.2, 64.0, 60.8, 60.7, 34.0 (4C), 33.9 (2C), 33.8, 19.2, 19.1, 19.0 (4C), 18.9 (5C), 18.8 (2C), 18.5.

MALDI-TOF MS: Calc [M+Na]⁺: 855.41. Found: 855.20.

6′-(Cyanine 7.5)-Isobutyric Sucrose (Sucrose Septaisobutyrate Cy 7.5) (4)

6′-OH-isobutyric sucrose (3) (14 mg, 0.017 mmol) was dissolved in dry DCM (3 mL). Then, Cyanine 7.5 NHS ester (15 mg, 0.019 mmol) was added, followed by addition of triethylamine (10 μL, 0.072 mmol). The reaction was stirred at room temperature for 2 days. Then, TLC (Hexane:Ethyl acetate 3:1) showed full conversion. The organic phase was concentrated in vacuo, the compound redissolved in methanol (2 mL) and purified by preparative HPLC (Column: Xterra C8. Eluent system: A: 0.1% TFA in water. B: Acetonitrile, 0.1% TFA. Gradient: 75-100% B within 15 minutes). Yield: 15.2 mg (62%). Texture: green powder. ¹H-NMR (800 MHz, DMSO-d₆): δ 8.24 (dd, J=11.5, 8.5 Hz, 2H), 8.09-8.02 (m, 4H), 7.84-7.63 (m, 6H), 7.53-7.47 (m, 2H), 6.19 (dd, J=26.1, 14.0 Hz, 2H), 5.62 (d, J=3.6 Hz, 1H), 5.50 (d, J=7.4 Hz, 1H), 5.40-5.31 (m, 2H), 5.10-5.06 (m, 1H), 4.93-4.85 (m, 2H), 4.33-4.01 (m, 8H), 3.86 (t, J=6.8 Hz, 1H), 3.76-3.74 (m, 2H), 2.93 (q, J=6.7 Hz, 1H), 2.59-2.53 (m, 10H), 2.47-2.30 (m, 3H), 2.07 (s, 3H), 1.94 (br s, 6H), 1.89-1.85 (m, 2H), 1.80-1.74 (m, 2H), 1.66-1.34 (m, 8H), 1.27-0.83 (m, 42H).

MALDI-TOF MS: Calc [M+H]⁺: 1464.78. Found: 1464.73.

Conclusion:

In conclusion, the product SSIB-Cy7.5 was formed in high yield and purity.

### Example 4: Preparation of SSIB-Cy7.5 Marker Formulations

In the current example, the NIR dye SSIB-Cy7.5 was dissolved in marker formulations based on LOIB or SAIB.

Methods:

Preparation of SAIB marker formulations: SAIB was heated to 70° C., and 7 g of SAIB was poured into a glass vial. 1 g of xSAIB and 2 g of EtOH was mixed with SAIB and sonicated for 30 minutes to obtain a transparent and homogeneous SAIB formulation (SAIB:xSAIB: EtOH 70:10:20).

Preparation of LOIB marker formulations: 7 g of LOIB was weighed into a glass vial. 1 g of xSAIB and 2 g of EtOH was mixed with SAIB and sonicated for 30 minutes to obtain a transparent and homogeneous LOIB formulation (LOIB:xSAIB: EtOH 70:10:20).

Addition of SSIB-CY7.5: A 2 mg/ml stock solution of SSIB-CY7.5 was prepared in EtOH. Afterwards, 0.1 ml SSIB-Cy7.5 stock solution was pipetted into a glass vial, and dried at 55° C. under a gentle stream of N₂. 2 g of either the SAIB or LOIB formulation was added, and the solution was sonicated for 6h, to obtain a final dye concentration of 0.01% w/w. Formulations containing 0.01%, 0.006%, 0.003%, 0.001%,0.0006%, 0.0003%, 0.0001% or 0.00001% SSIB-Cy7.5 were prepared by serial dilution.

Addition of D&C violet 2 (D&Cv2): 0.01% (w/w %) of SSIB-Cy7.5 and 0.1% (w/w %) blue D&C violet 2 in LOIB markers was prepared for testing in animals. Briefly, 1 mg D&C violet 2 was mixed in 1 g of SSIB-CY7.5—LOIB marker solution (LOIB:xSAIB:EtOH:SSIB-Cy7.5 70:10:20:0.01) and the solution was sonicated for 30 min.

Results:

NIR marker formulations based on either LOIB or SAIB were prepared containing 10% w/w xSAIB, 20% EtOH and 0.00001%-0.01% w/w SSIB-Cy7.5. The formulations were transparent and homogenous and could be stored at 5° C. without precipitation or change in appearance for more than 6 months. The formulations containing 0.1% w/w D&C violet 2 were dark blue and prepared for animal testing.

Conclusion:

In conclusion, transparent and homogeneous NIR marker formulations could be prepared containing the novel NIR dye SSIB-Cy7.5.

### Example 5: Preparation of PC1, PC2 and PC3 Marker Formulations

In the current example, the NIR dyes PC1, PC2 and PC3 were formulated in marker formulations.

Methods:

Preparation of SAIB marker formulation: SAIB was heated to 70° C., and 7 g of SAIB was poured into a glass vial. 1 g of xSAIB and 2 g of EtOH was mixed with SAIB and sonicated for 30 minutes to obtain a transparent and homogeneous SAIB:xSAIB:EtOH formulation (SAIB:xSAIB:EtOH 70:10:20). 8 g of SAIB was poured into a glass vial. 2 g of benzyl alcohol (BA) was mixed with SAIB and sonicated 30 minutes to obtain a transparent and homogeneous SAIB:BA formulation (SAIB:BA 80:20).

Preparation of SAIB Marker Formulations with Dyes:

PC1: A solution of PC1 dissolved in chloroform (50-500 μL, 1 mg/mL) was pipetted into a glass vial, and the chloroform was evaporated at room temperature under nitrogen flow. Subsequently, 1 g of marker formulation (SAIB:xSAIB:ethanol 70:10:20) was added into the vial to achieve a PC1 concentration of 0.005-0.05% w/w. The resulting mixture was sonicated at 70° C. for 15 minutes followed by vortexing.

PC2: 1 mg of PC2 was weighed into a glass vial. 1 g of SAIB marker formulation was added, and the solution was sonicated for at 55° C. 6 h and followed by magnetically stirring at 55° C. for 16 hours, to obtain a final dye concentration of 0.1% w/w.

PC3: A solution of PC3 dissolved in chloroform (50-500 μL, 1 mg/mL) was pipetted into a glass vial, and the chloroform was evaporated at room temperature under nitrogen flow. Subsequently, 1 g marker formulation (SAIB:BA 80:20) was added into the vial to achieve a PC3 concentration of 0.005-0.05%. The resulting mixture was sonicated at 70° C. for 15 minutes followed by vortexing.

Formulations containing differing dye contents were produced by serial dilution using marker solution followed by vigorous stirring.

Conclusion:

In conclusion, all three phthalocyanine dyes were successfully dissolved in marker formulations containing SAIB.

### Example 6: Spectroscopic Characterization of the PC1, PC2 and PC3 NIR Dyes

In the current example, the absorbance and fluorescence of the phthalocyanine based NIR dyes, PC1, PC2 and PC3 were investigated when dissolved in toluene and in marker solution.

Methods:

Preparation of PC dyes in organic solvents: Stock solutions of all the PC dyes in toluene were prepared (1 mg/mL) by simple mixing, and further diluted to suitable concentration for absorption and fluorescence measurement. Three concentrations of each dye were prepared in toluene and used for recording of fluorescence spectra and one dye concentration was prepared for absorbance.

Preparation of PC dyes in marker formulations: All compositions are represented as weight percent or weight ratios. Marker formulations, containing SAIB (70%), xSAIB (10%) and EtOH (20%), or SAIB (80%) and benzyl alcohol (BA) (20%), were prepared as describe in example 5.

A PC1 marker solution based on SAIB:xSAIB:ethanol 70:10:20 was prepared as described in example 5. The PC1 marker solution was further diluted with SAIB:xSAIB:ethanol 70:10:20 to a suitable concentration for absorption (0.001% w/w) and fluorescence measurement (0.005% w/w).

A PC2 marker solution based on SAIB:xSAIB:ethanol 70:10:20 was prepared as described in example 5. The PC2 marker solution was further diluted with SAIB:xSAIB:ethanol 70:10:20 to a suitable concentration for absorption (0.001% w/w) and fluorescence measurement (0.001% w/w).

A PC3 marker solution based on SAIB:BA 80:20 was prepared as described in example 5. The PC3 marker solution was further diluted with SAIB:BA 80:20 to a suitable concentration for absorption (0.01% w/w) and fluorescence measurement (0.005% w/w).

Fluorescence emission measurements: Each marker formulation (1.0 mL) was transferred to a quartz cuvette (Helma, 10 mm light path), and the fluorescence spectrum was collected by a fluorescence spectrometer (OLIS DM 45) with excitation/emission bandwidth of 26 nm and integration time of 0.2 seconds. An excitation wavelength of 650 nm was used for PC1 in toluene and the marker formulation. For PC2, an excitation wavelength of 700 nm was utilized for the dye in toluene and the marker formulation, where as an excitation wavelength of 800 nm was utilized for PC3 in toluene and 750 nm for PC3 in the marker formulation.

UV-vis absorbance measurements: Each solution (0.2 mL) was pipetted into a 96-well plate, and the UV-vis spectrum (400-1000 nm) was recorded by a multimode microplate reader (Spark®, Tecan) with bandwidth of 3.5 nm.

Results:

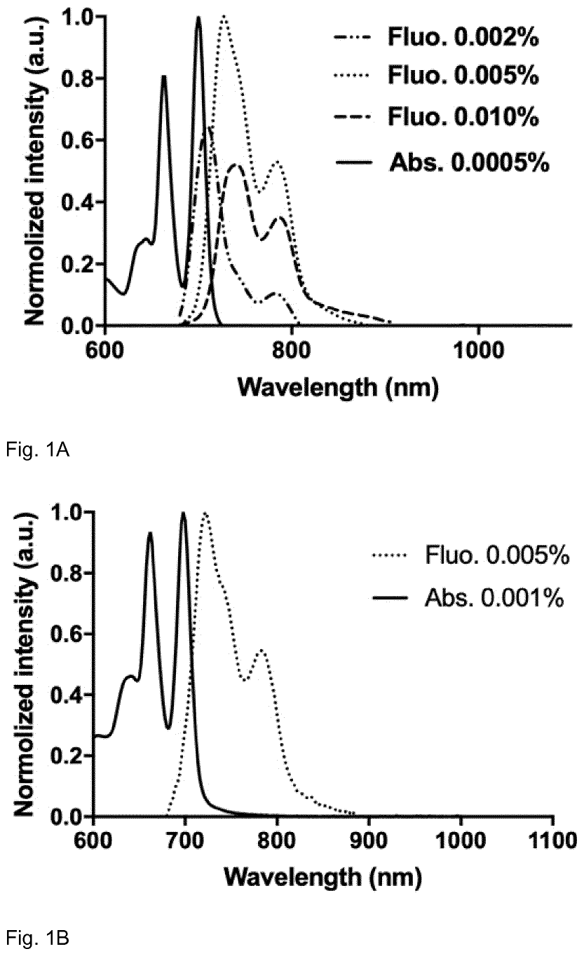

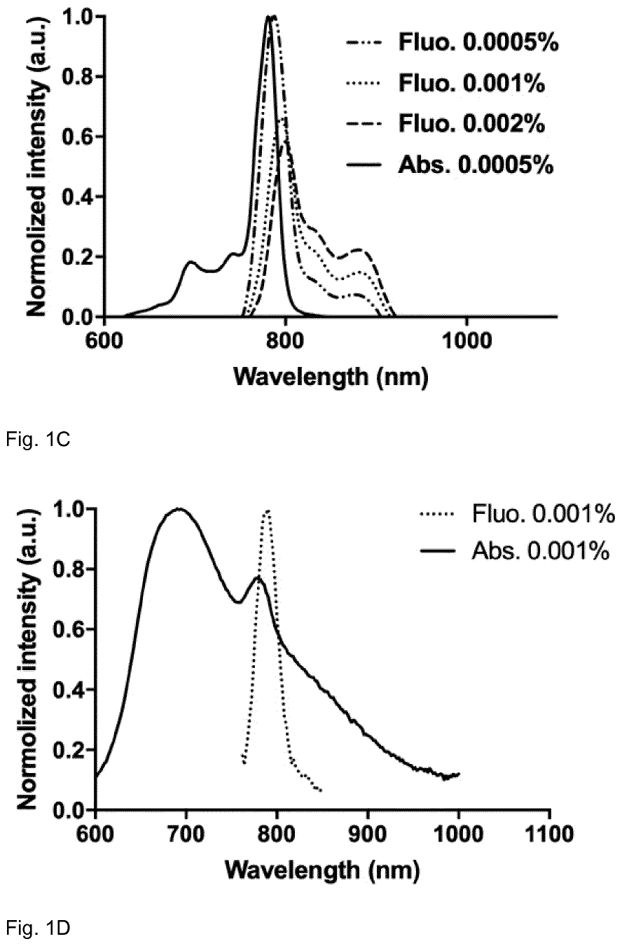

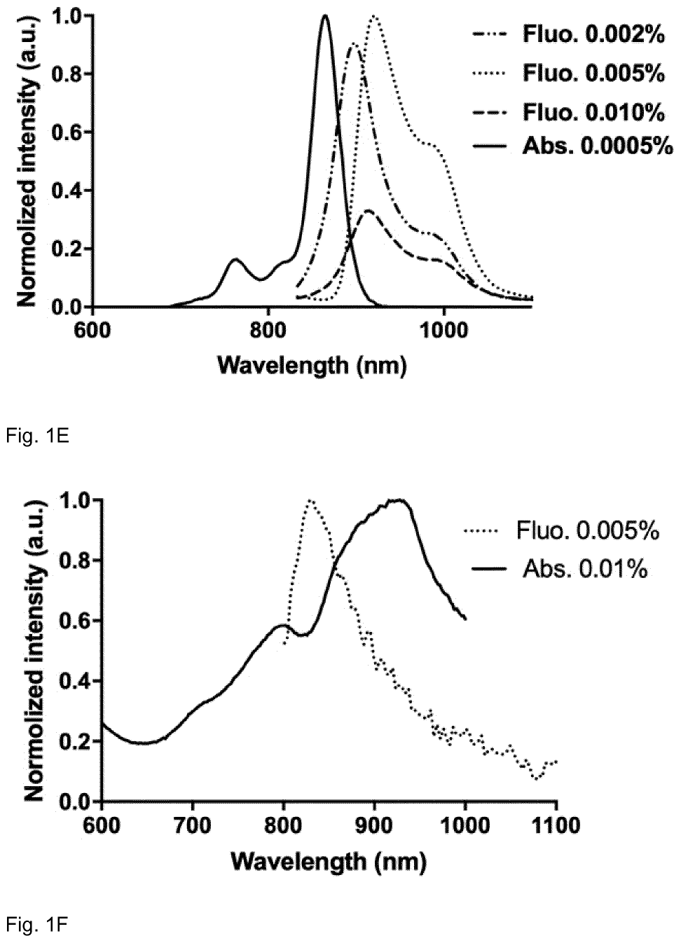

The absorbance and fluorescence of the three phthalocyanine dyes PC1, PC2 and PC3 were investigated and the results are presented in FIG. 1.

Conclusion:

In conclusion, PC1 was found to fluoresce in the 700-800 nm range and absorb light from 600-700 nm. PC2 displayed a sharper fluorescence band centred about 790 nm with a shoulder towards higher wavelengths in toluene. In marker formulation, PC2 displayed a narrow emission peak centred about 790 nm. PC2 absorbed light in the range 650-800 nm range with a sharp peak at 782 nm in toluene. A broader absorption band was observed for PC2 when embedded in the SAIB:xSAIB:EtOH 70:10:20 formulation. In toluene, PC3 fluoresced in the range 850-1100 nm and absorbed light in the 700-900 nm range. When dissolved in SAIB:BA 80:20, PC3 displayed broader absorption and emission peaks compared PC3 dissolved in toluene. All three PC dyes displayed fluorescence emission intensity dependence on the fluorophore concentration indicating self-quenching.

### Example 7: Fluorescence Self-Quenching Analysis of Phthalocyanines

In order to identify the brightest marker formulation with the highest fluorescence intensity, the self-quenching of the PC2 dye was investigated. The self-quenching was investigated by UVvis, fluorescence collected at 90° using a cuvette, and surface fluorescence collected via a surface plate reader.

Method:

Formulations with the composition SAIB:x-SAIB:EtOH 70:10:20 containing different levels of PC2 were prepared as described in example 5.

Fluorescence emission by fluorometer: Briefly, the fluorescence emission intensity of marker formulation samples containing either 0.01%, 0.006%, 0.003%, 0.001%, 0.0006%, 0.0003%, 0.0001% or 0.00001% PC2 dye were recorded, e.g. 1.2 mL of the PC2 formulations were pipetted into quartz cuvettes (Helma 10.00 mm), and the fluorescence emission was recorded from 780 nm to 830 nm using a fluorescence spectrometer (OLIS SLM8000, USA). The emission spectra were recorded using an excitation wavelength of 768 nm, scan-time of 45s, and a slit-width of 8 nm.

Surface fluorescence imaging: The surface fluorescence of the gel samples was investigated as function of PC2 dye concentration using an in vitro NIR imaging system (Odyssey FC, Licor, USA). 70 uL of gel sample with different PC2 dye concentration was pipetted onto a 10-well specimen glass (Thermo Scientific, 10-well 6.7 mm), and the fluorescence was recorded using the 800 nm channel setting (785 nm excitation, resolution of 125 μm). The 10-well specimen-glass with samples was subsequently kept in a vacuum oven at 55° C. overnight to remove EtOH. After EtOH removal, the samples were cooled to room temperature and the fluorescence was remeasured.

Results:

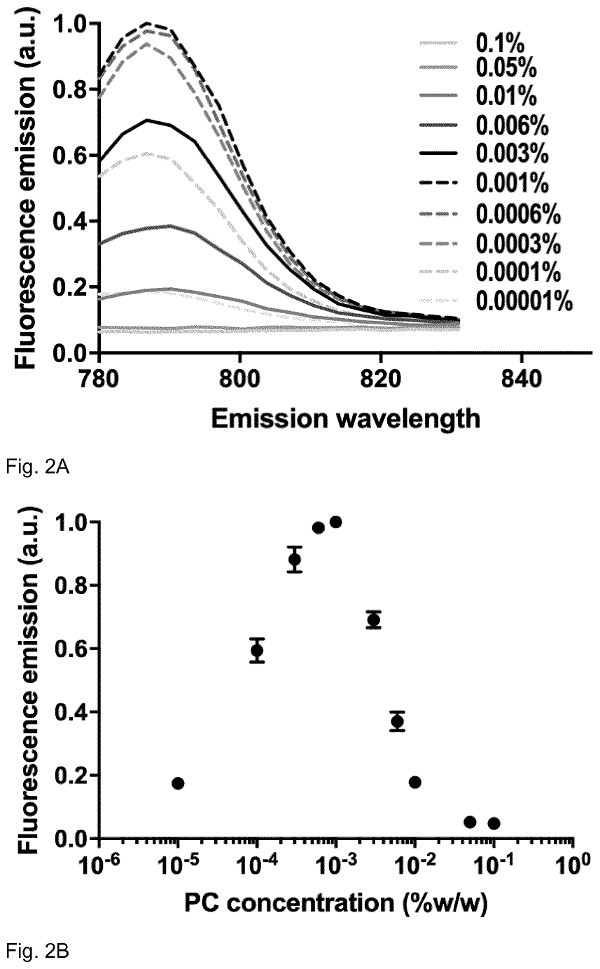

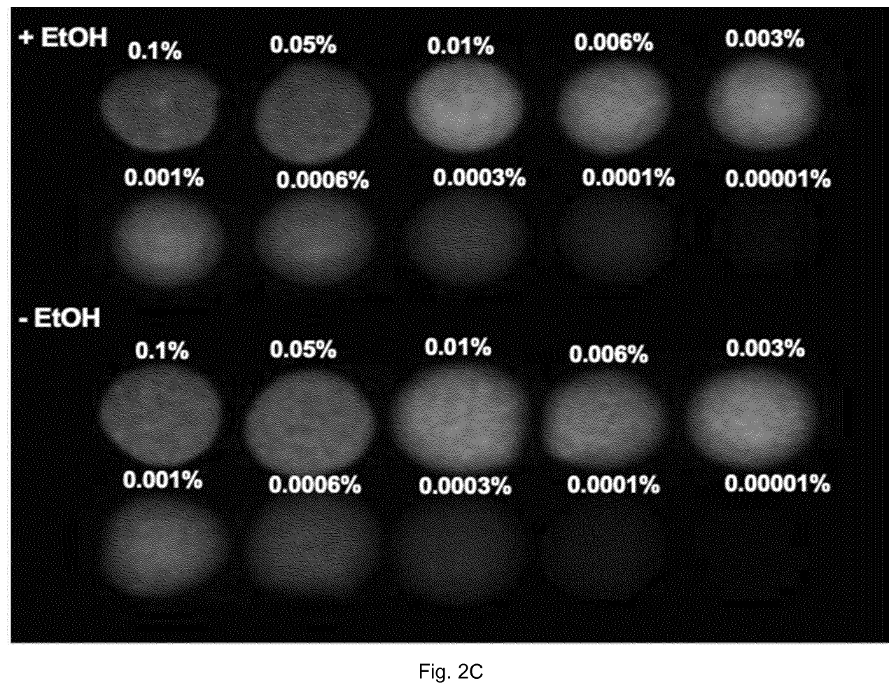

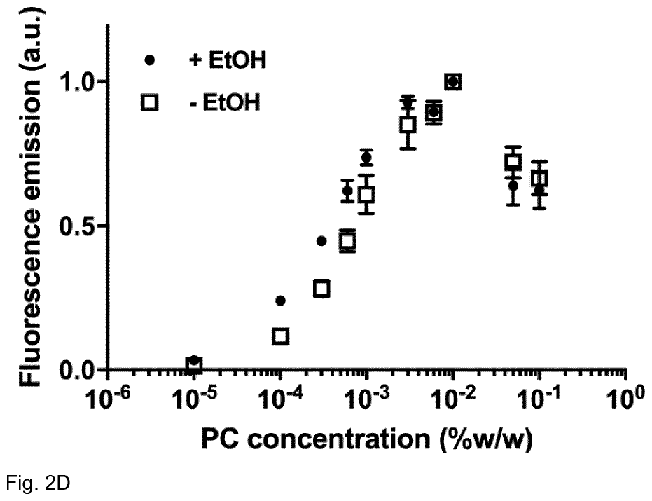

The fluorescence self-quenching of the phthalocyanine dye PC2 was investigated using standard 90° cuvette measurements and via surface fluorescence imaging, and the results are presented in FIG. 2.

Conclusion:

In conclusion, the fluorescence intensity of phthalocyanine dye PC2 formulated in SAIB:x-SAIB:EtOH 70:10:20 was found to depend on the dye concentration in both standard cuvette and in surface fluorescence assays. The maximum intensity and lowest degree of self-quench, was determined to 0.001% w/w in standard emission fluorescence and 0.01% w/w in surface fluorescence. The self-quenching was found not to depend on EtOH release, i.e. the marker has the same fluorescence intensity before and after EtOH efflux.

### Example 8: Spectroscopic Characterization of the SSIB-Cy7.5 Dye

The novel NIR dye SSIB-Cy7.5 was formulated in markers based on SAIB or LOIB and characterized by fluorescence or absorbance. The fluorescence emission was furthermore investigated as function of dye concentration in SAIB based markers.

Methods:

The marker formulations SAIB:xSAIB:EtOH and LOIB:xSAIB:EtOH:D&Cv2 containing SSIB-Cy7.5 were prepared according to example 4.

Fluorescence spectrum: For SAIB:xSAIB:EtOH SSIB-Cy7.5 formulations, 1 mL of samples were pipetted into quartz cuvettes (Helma 10.00 mm), and the fluorescence emission from 780 nm to 900 nm was recorded using a fluorescence spectrometer (OLIS SLM8000, USA). The emission spectra were recorded using an excitation wavelength of 768 nm, scan-time of 45s, and a slit-width of 8 mm. For the LOIB:xSAIB:EtOH SSIB-Cy7.5 formulations, 1 mL of samples were pipetted into quartz cuvettes (Helma 10.00 mm), and the fluorescence emission from 800 nm to 1100 nm was recorded using a fluorescence spectrometer (OLIS DM45, USA) with excitation/emission bandwidth of 26 nm and integration time of 0.2 seconds. An excitation wavelength of 768 nm was employed.

The UVvis spectra of SSIB-Cy7.5 marker sample was recorded using a multimode microplate reader (Tecan, Sweden), e.g. 0.2 mL of the SSIB-Cy7.5 formulations 0.05 mL of the SSIB-Cy7.5 formulation with D&Cv2 were pipetted into 96-well plate, and the UV-vis spectra from 550 nm to 1000 nm was measured.

Results:

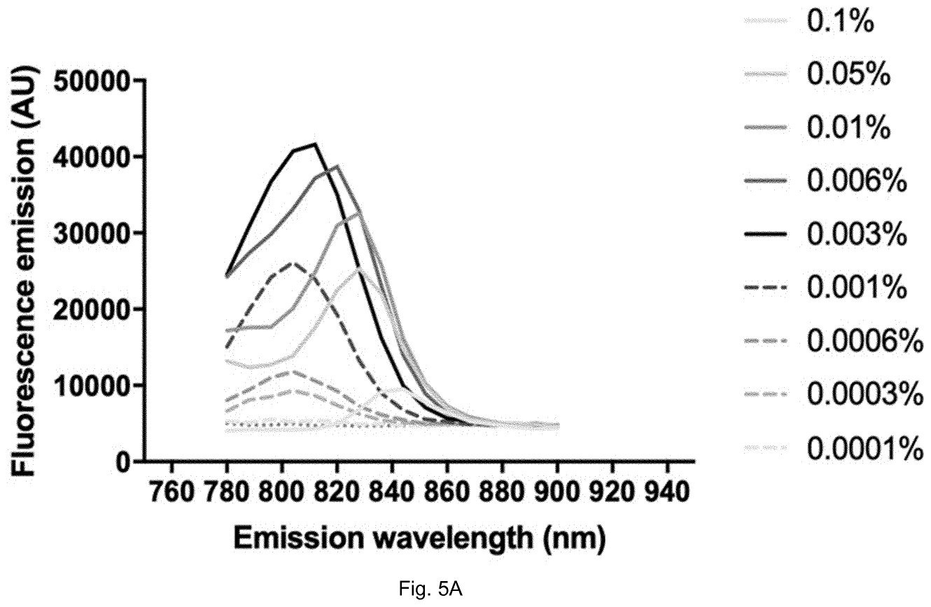

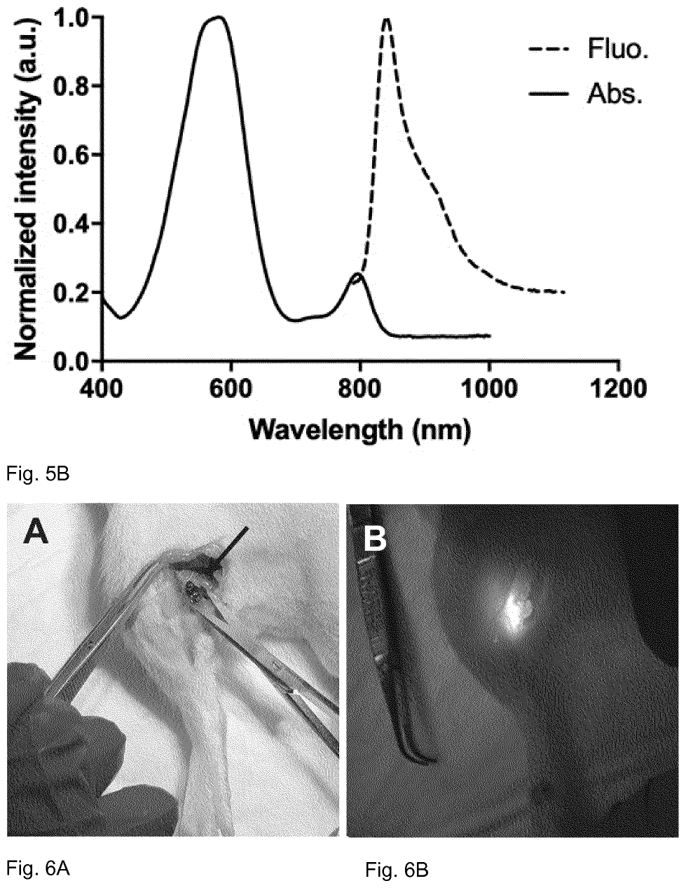

Fluorescent markers containing SSIB-Cy7.5 were prepared and spectroscopically characterized. The results are presented in FIG. 5. The fluorescence emission of SSIB-Cy7.5 in SAIB:xSAIB:EtOH 70:10:20 displayed a dye concentration dependent change in the emission intensity and a gradual shift in peak intensity from 804 to 844. As the concentration of SSIB-Cy7.5 was increased, the emission intensity increased until 0.003% w/w after which it decreased indicating dye self-quenching. UVvis analysis of LOIB:xSAIB:EtOH:D&Cv2 displayed absorption peaks at 590 nm and 803 nm corresponding to absorption from the 0.1% w/w D&Cv2 dye and 0.01% w/w SSIB-Cy7.5 dye respectively. A broad emission peak centred at 845 nm was determined from 0.01% w/w SSIB-Cy7.5 in LOIB:xSAIB:EtOH:D&Cv2.

Conclusion:

In conclusion, SSIB-Cy7.5 was successfully formulated in both LOIB and SAIB marker formulations, and displayed both fluorescence intensity and peak position dependence on the dye concentration.

### Example 9: In Vitro Leaching of PC2 from a Marker

Upon injection of the markers in aqueous media or tissues, EtOH diffuses from the marker, which may lead to leaching of the dye. This phenomenon was investigated in vitro by injecting a marker formulation containing a high amount (0.1% w/w) of PC2 into a phosphate buffered solution. The leaching of dye from the marker was afterwards detected by UVvis spectroscopy.

Methods:

The marker formulation SAIB:xSAIB:EtOH 70:10:20 was prepared containing 0.1% w/w PC2 as described in example 4-5 and used for the leaching experiment.

In vitro leaching of PC2 dye from the marker: The release of PC2 dye from the marker was investigated in vitro, by injecting 300 μL of PC2 marker formulation (0.1%˜1 mg/ml) into 5 mL of phosphate buffer saline (PBS, 5 mM, 150 mM NaCl, pH 7.0). The sample was afterwards stored in the dark at 37° C., and dye release was monitored by UV-vis spectroscopy after 1, 3, 6 hour and 1, 2, 4, 6 days. UV-vis spectra of 0.5 ml PBS release buffer was recorded in quartz cuvettes from 600 nm to 850 nm using a Nanodrop 2000c

(Thermoscientific, US) spectrophotometer. Due to the low solubility of PC2 in buffer, the standard curve of PC in buffer could not be achieved. A PC2 solution in acetonitrile (0.05 mg/mL) was prepared, and diluted using PBS buffer to the concentration of 0.006 mg/mL corresponding to 10% release of PC2 in PBS.

Results:

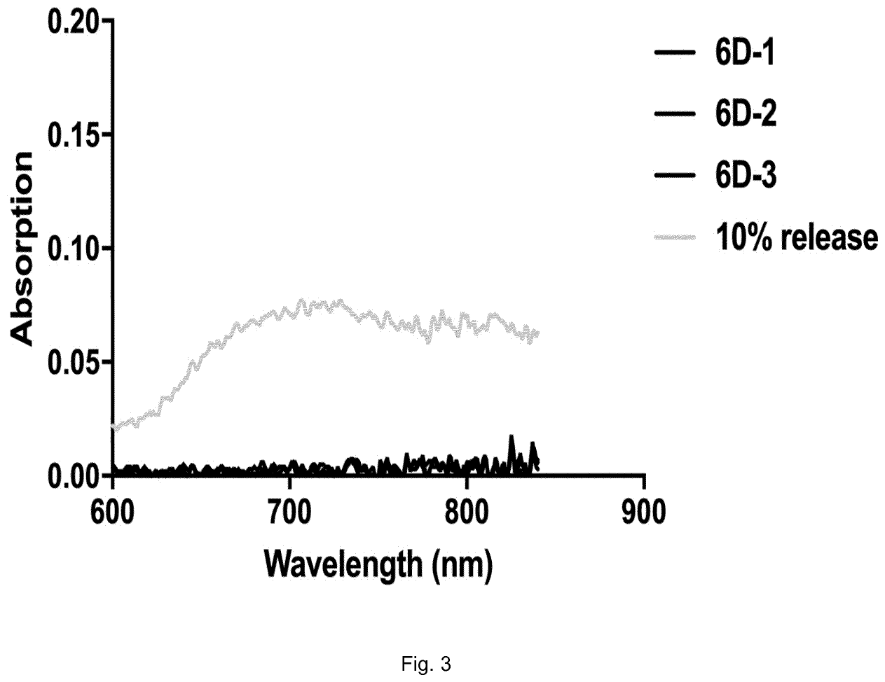

The formulation containing SAIB:xSAIB:EtOH 70:10:20 and PC2 dye was prepared, and the PC2 in the release media was determined by UVvis (FIG. 3).

No change in absorbance in the release media was determined within 6 days, indicating that the dye is fully retained in the formed marker. Based on the included 10% standard (FIG. 3), less than 1% of the PC2 dye is released. The latter result is likely lower than 1%, but cannot be resolved with the current baseline noise level.

Conclusion:

In conclusion, no leakage of PC2 from the marker is detected within the 6 day timeframe of the experiment.

### Example 10: Fluorescence Quenching of PC2 Due to Chelation of Copper

The phthalocyanine dyes are chelators and may coordinate metal cations and changes in the electronic properties of the chelate may cause changes in fluorescence and absorbance of the dye.

Methods:

The marker formulation SAIB:xSAIB:EtOH 70:10:20 was prepared containing 0.001% w/w PC2 as described in example 4-5.

Copper quenching samples: A solution of CuCl₂.2H₂O in ethanol (0.005 mg/mL) was prepared and transferred to glass vials (0, 44, 87, 131, 218 or 436 μL). The ethanol in each vial was evaporated by heating to 55° C. using nitrogen flow. PC2 marker solution (1.2 mL, 0.001%) was added to each vail containing different amount of CuCl₂, and the molar ratio of Cu²⁺/PC2 in each vial was afterwards 0, 1:10, 1:5, 3:10, 1:2 and 1:1, respectively. The resulting mixtures were magnetically stirred at 55° C. for 2 hours.

The UVvis spectra of PC2 marker samples were recorded using a multimode microplate reader (Tecan, Sweden), e.g. 0.2 mL of the PC2 formulations were pipetted into 96-well plate, and the UV-vis spectra from 550 nm to 1000 nm was measured.

The fluorescence emission spectra of each mixture were obtained. Briefly, each marker solution (1.2 mL) was transferred to a quartz cuvette and the fluorescence emission spectra was recorded in a wavelength range of 780-830 nm at an excitation wavelength of 768 nm, scan time of 45 seconds and a slit width of 8 mm.

Results and Conclusion:

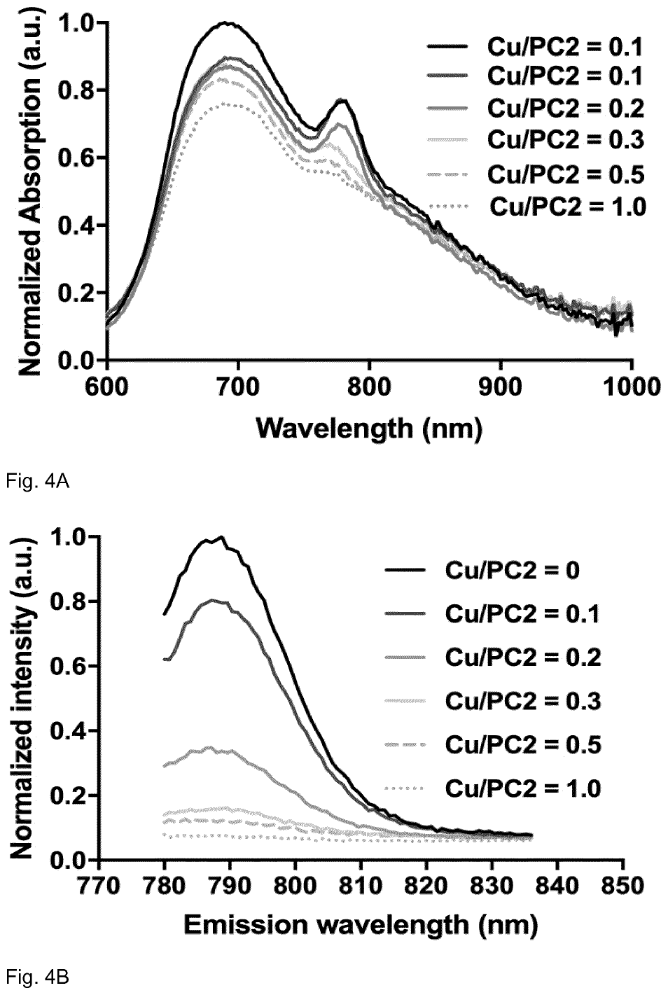

Based on the data presented in FIG. 4, it is concluded that copper is chelated by PC2, and that the fluorescence intensity of PC2 is reduced upon binding of copper. A 1:1 molar ratio of dye and copper is required to fully quench the PC2 dye.

### Example 11: Radiolabelling of Phthalocyanine Class Dyes with ⁶⁴Cu

The phthalocyanine class dyes (PC1, PC2 and PC3) are metal chelators as demonstrated in example 10, where copper quenched the fluorescence emission of PC2. In this example, marker formulations containing PC2 are radiolabelled with ⁶⁴Cu²⁺ and afterwards quantified by Radio-TLC.

Method:

⁶⁴Cu production: ⁶⁴Cu was produced on a PETtrace cyclotron (GE Healthcare) equipped with a beamline by proton irradiation of an electroplated ⁶⁴Ni target, then purified by anion exchange chromatography in aqueous hydrogen chloride (HCl) media. The ⁶⁴Cu was ultimately obtained in aqueous HCl (1.0 M), and isolated by evaporation of aqueous HCl by argon flow. The dry ⁶⁴CuCl₂ was used for radiolabelling markers.

Radiolabelling of markers: A marker SAIB:xSAIB:EtOH (70:10:20) containing PC2 (750 μL, 0.01% or 0.001%), or a marker without PC2 (750 μL) was added to dry ⁶⁴CuCl₂ (150 MBq) in a glass vial. The resulting mixtures were magnetically stirred at 55° C. for 2 hours.

Radio-TLC characterization: A small amount of each radiolabelling markers was weighed into glass vial and dissolved in acetonitrile to a concentration of about 10 mg/mL. The resulting solution was analysed by radio-TLC (Perkin-Elmer, MiniGita Star with a Beta Detector GMC probe) by spotting 1 μL onto a TLC plate (Merck, silica gel 60 F254). The TLC plates were developed using chloroform:methanol:mili-Q water:acetic acid 70:25:4:1 (v/v) as eluent. Non-complexed ⁶⁴Cu is known to stay at the origin using these TLC conditions.

Results:

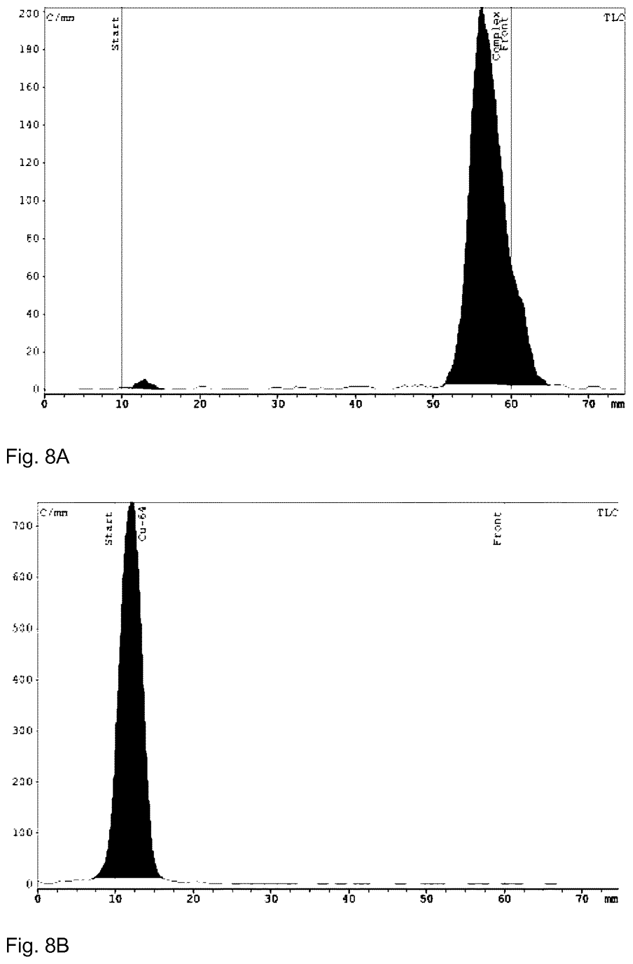

The marker SAIB:xSAIB:EtOH:PC2 (70:10:20:0.01) was radiolabelled with ⁶⁴Cu and the complex formation was investigated using Radio-TLC. The data are presented in FIG. 8. The formation of ⁶⁴Cu-PC2 was confirmed by comparing the obtained TLC retention factor (Rf=0.9-1.0) with that of a non-radioactive chemically identical reference compound. The Rf of ⁶⁴Cu in marker without PC2 remained at the origin (Rf=0).

Conclusion:

In conclusion, PC2 readily complexes ⁶⁴Cu by direct mixing of SAIB:xSAIB:EtOH:PC2 (70:10:20:0.01) and dry ⁶⁴CuCl₂. With the presence of 0.01% w/w PC2 in the marker formulation, >99% of ⁶⁴Cu moved with the complex at the solvent front on the TLC-plate.

### Example 12: In Vitro Release and Transfer Efficiency of ⁶⁴Cu Labelled Markers

The transfer efficiency and in vitro release of ⁶⁴Cu from radiolabelled PC2

SAIB:xSAIB:EtOH 70:10:20 markers were investigated. An iso-osmotic TRIS buffer containing EDTA as scavenger of free ⁶⁴Cu²⁺ and liposomes as scavenger of released PC2 dye was used as in vitro release media.

Methods:

Marker preparation: Radiolabelled SAIB:xSAIB:EtOH 70:10:20 markers containing PC2 (100 μL, 0.01% or 0.001%) and a control marker without PC2 (100 μL) were prepared as described in example 5.

Preparation of stealth liposomes: The stealth liposomes (HSPLC:Chol:DSPE-PEG2k 3:1:1 w/w) were produced by hydration of a commercial stealth lipid mixture with iso-osmotic TRIS buffer at 65° C. by sonication for 1 hour, followed by sizing with a mini-extruder equipped with 200 nm polycarbonate filter. The liposome size was 142.4±1.6 nm with a PDI of 0.19±0.006. The lipid concentration was determined using ICP-MS and the liposomes were further diluted by ISO-TRIS to a final concentration of 5 mM.

In vitro release assay: Radiolabelled markers were afterwards injected through a 25G needle into a glass vial containing release medium (4.0 mL) containing TRIS (10 mM, 150 mM NaCl, pH 7.8) buffered EDTA (1.0 mM) and stealth liposomes (5.0 mM lipids). The radioactivity of each marker injected into release buffer was measured on a dose calibrator (Comecer, VDC-505). Aliquots (15-1000 μL) were removed as a function of time (1 hour, 3 hours, 6 hours, 1 day, 2 days, 4 days and 6 days), and replaced with an equal amount of release medium. After 6 days, all the release medium was removed and the remaining marker was dissolved using ethanol (1.0 mL). An aliquot of the resulting solution (250 μL) was removed for quantification. All sample aliquots were subsequently mixed with LSC cocktail (Ultima Gold), and were analysed by liquid scintillation (HIDEX, 300 SL spectrometer) with the energy range of 2-850 keV. A calibration curve (20-800 Bq) was prepared for ⁶⁴Cu, which was linear in the required concentration range (r²>0.999).

Transfer efficiency: The transfer efficiency measures the fraction of the total activity that has been solubilized in the sample. The transfer efficiency was determined for the individual formulations by determination of the activity concentration, i.e. 100 μL sample was transferred to a glass vial, and the activity was determined by dose-calibrator (Comecer, VDC-505).

Results:

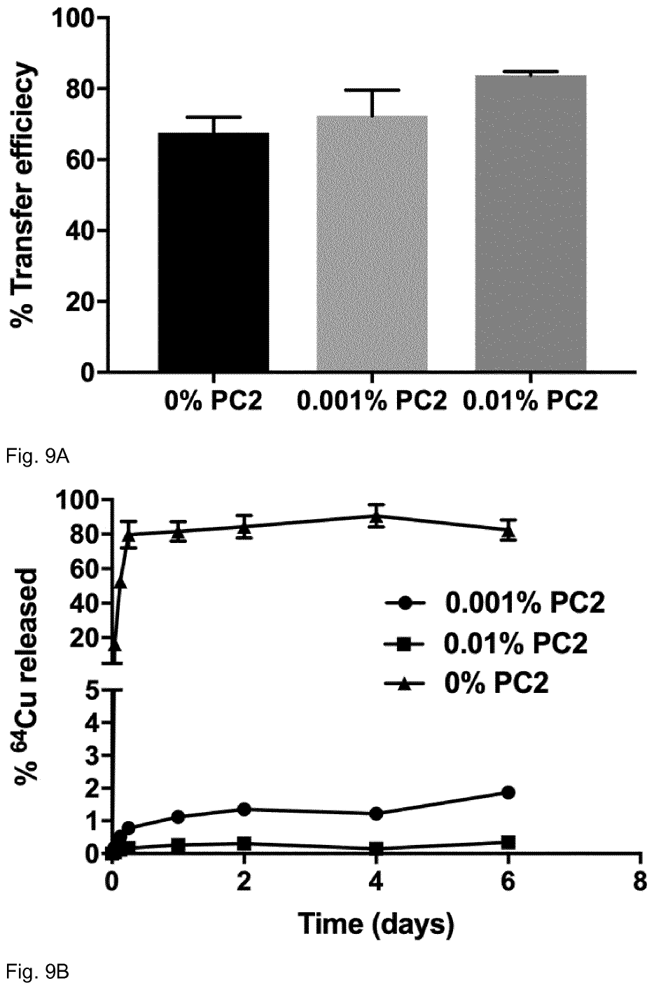

Markers were successfully prepared and radiolabelled, and the in vitro release and transfer efficiency was determined. The results are reported in FIG. 9.

The SAIB:xSAIB:EtOH 70:10:20 marker formulations containing either 0, 0.001 or 0.01% w/w PC2 were radiolabelled with ⁶⁴Cu, and an increasing transfer efficiency was observed with increasing PC2 dye concentration, which confirms that copper is chelated by PC2. Without presence of PC2 (0% w/w), 67% of the ⁶⁴Cu was solubilized in the marker solution indicating an affinity of copper for the oxygen-rich marker constituents SAIB, xSAIB and EtOH.

Significant differences were observed between the markers in the in vitro release assay, where the marker containing no PC2 (no chelator) displayed a rapid burst, releasing 80% of the activity within hours. Markers containing 0.001% or 0.01% w/w PC2 exhibited limited release of less than 2% or less than 0.4% respectively. The latter result confirms that PC2 and copper forms a chelate as higher dye/chelator concentration leads to higher radionuclide retention in the marker as expected.

Conclusion:

In conclusion, markers containing PC2 can be readily radiolabelled by ⁶⁴Cu with a high transfer efficiency (>80%) and low degree of in vitro release (<2%).

### Example 13: NIR Guided Surgery Using the Marker LOIB:xSAIB:EtOH:D&Cv2:SSIB-Cy7.5

The formulation LOIB:xSAIB:EtOH:D&Cv2:SSIB-Cy7.5 was investigated as a surgical marker in a rat and porcine model using NIR image guidance.

Methods:

The marker formulation LOIB:xSAIB:EtOH:D&Cv2:SSIB-Cy7.5 70:10:20:0.1:0.01 was prepared as describe in example 4.

Rat model: A Wistar male rat (bodyweight 400 g) was euthanized by intravenous injection of an overdose of pentobarbital (Euthanimal Vet, 400 mg/ml, Scanvet, Hørsholm, Denmark) and 50 μL marker formulation was injected intramuscularly in the right thigh and in one testicle using a 1 ml syringe and a 23 G injection needle. The fluorescence of the markers was evaluated using a NIR camera (Fluobeam 800, Fluoptics, Grenoble, France), and were surgically exercised.

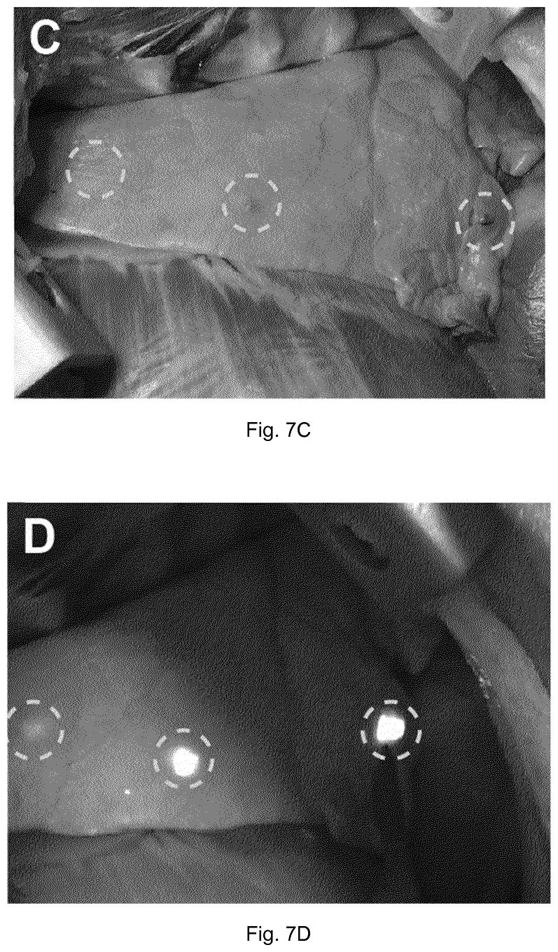

Porcine model: A 45 kg standard breed pig was euthanized by intravenous injection of an overdose of pentobarbital (Euthanimal Vet, 400 mg/ml, Scanvet, Hørsholm, Denmark) and the thoracic cavity opened by an incision through the thoracic wall. The incision wound was opened and maintained open using a large Wickers wound retractor (FIG. 7A). Marker was injected from the axial side of the lung at three depths to provide imaging data on marker performance in terms of fluorescence emission using a NIR camera (Fluobeam 800, 800 nm config, Fluoptics, Grenoble, France).

Results:

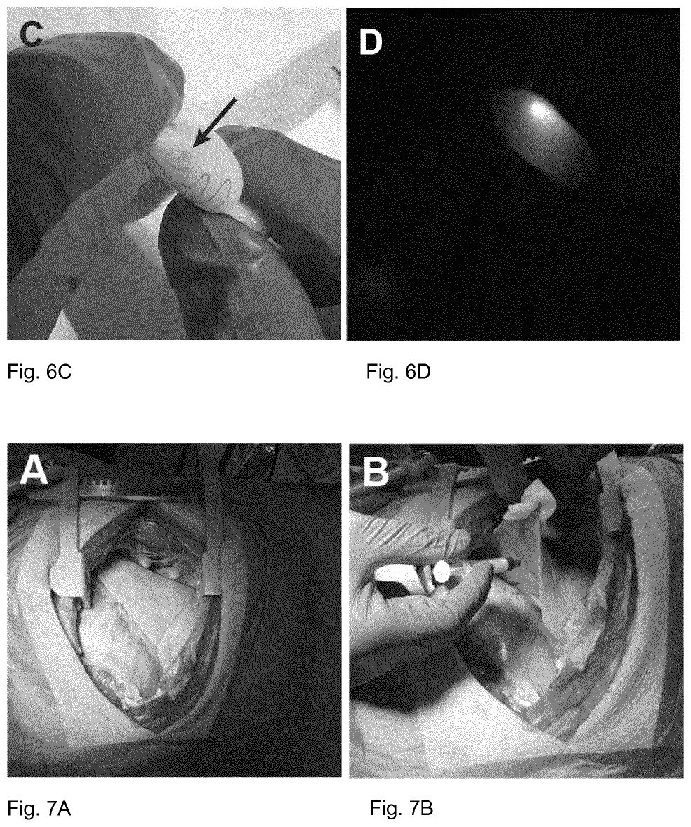

LOIB:xSAIB:EtOH:D&Cv2:SSIB-Cy7.5 markers were successfully injected and identified by NIR camera in the thigh and testicle of a rat (FIG. 6), and in porcine lung tissue (FIG. 7). In the rat model, the markers were visible during surgery (FIG. 6A) or when peripherally embedded in tissue (FIG. 6C) due to the D&Cv2 dye, but were more distinct when visualized using the NIR camera (FIGS. 6B and D). In the porcine model, the markers could be identified at a tissue depth up to −1 cm, but the emission light was increasingly attenuated in the deepest tissues (FIG. 7D, left marker). The LOIB:xSAIB:EtOH:D&Cv2:SSIB-Cy7.5 could in addition be identified visually via the blue D&Cv2 dye for peripheral/surface embedded markers (FIG. 7C, right marker).

Conclusion:

In conclusion, the SSIB-Cy7.5 dye enable NIR imaging of the LOIB:xSAIB:EtOH:D&Cv2 formulation allowing for localization of markers inside tissues.

### Example 14: In Vivo PET/NIR/CT Imaging of Markers in a Murine Model

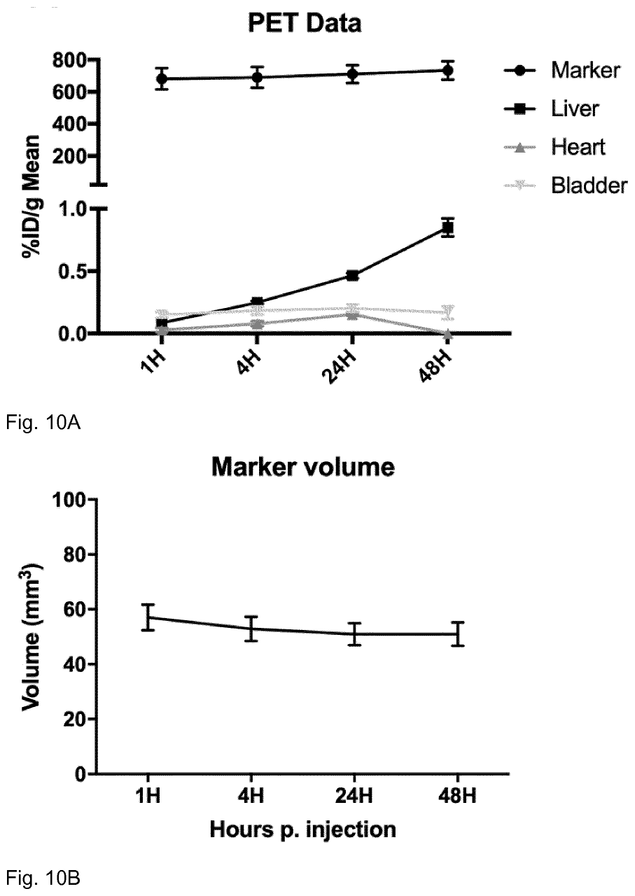

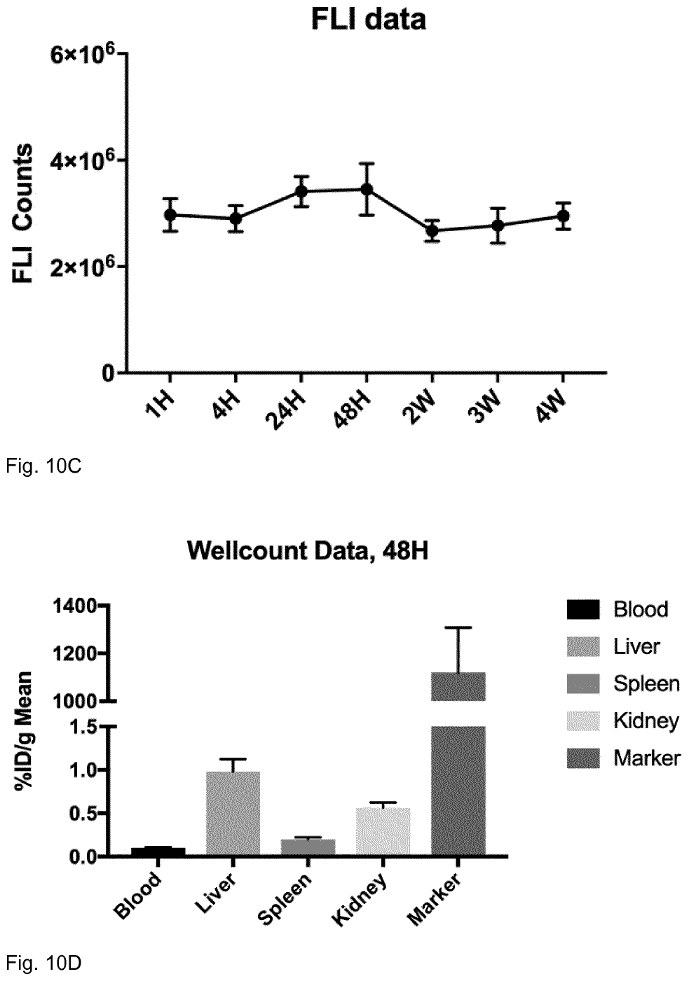

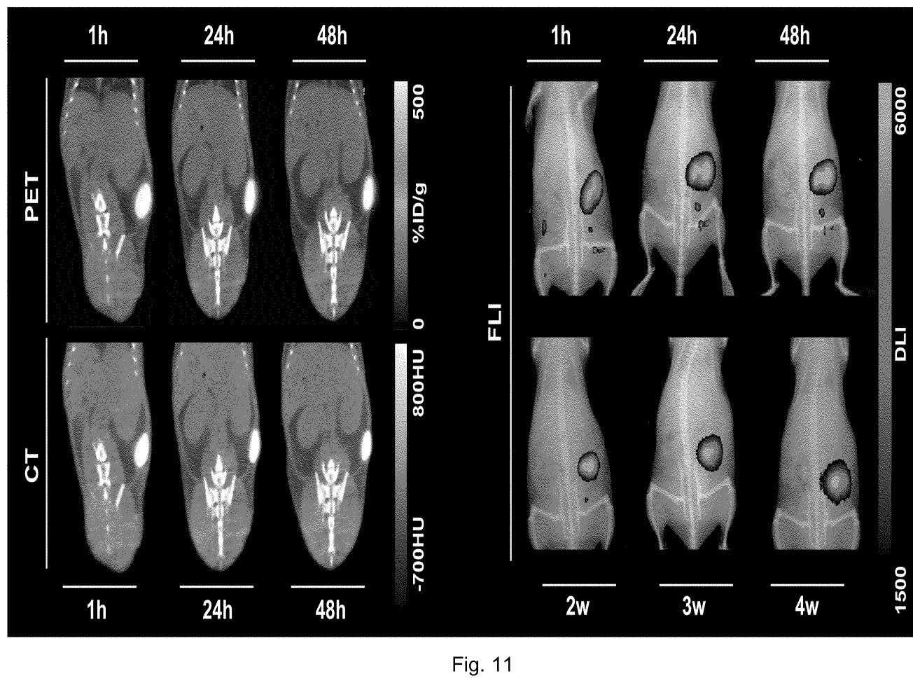

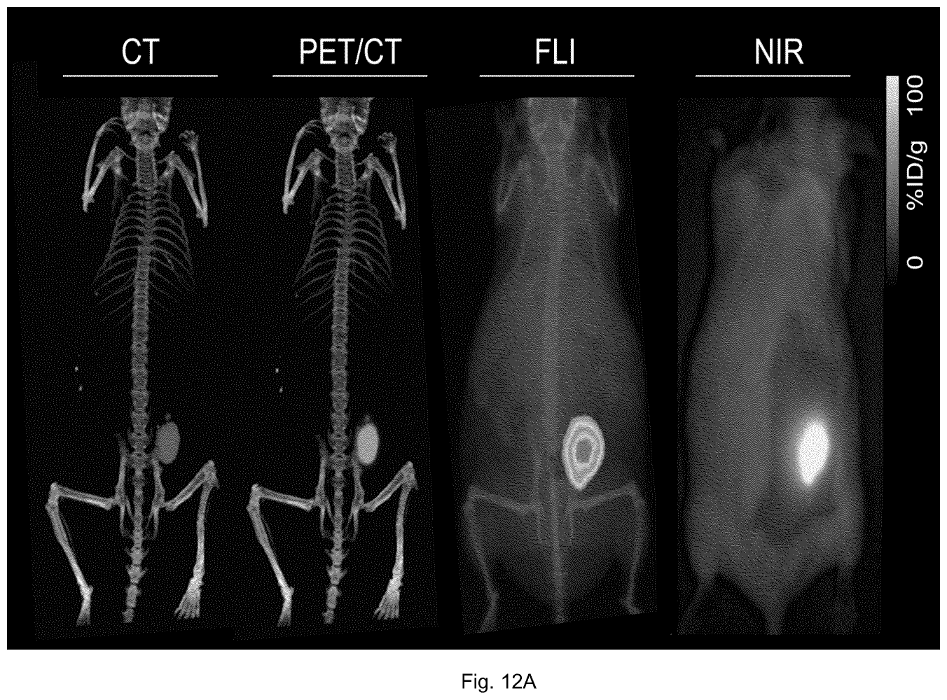

⁶⁴Cu radiolabelled SAIB:xSAIB:EtOH:PC2 70:10:20:0.01 markers were prepared and injected subcutaneously on the flank of 8 mice. The animals were afterwards PET/CT and fluorescence (FLI) 2D imaged using an IVIS scanner (Perkin Elmer). The data are compiled as quantitative measures in FIG. 10 and recorded images in FIG. 11.

Methods:

⁶⁴Cu radiolabelled SAIB:xSAIB:EtOH:PC2 70:10:20:0.01 markers were prepared as described in example 10. At the time of injection, the markers had an activity of 35 MBq/ml.

Study setup: 8 mice (NMRI/Taconic) were subcutaneously injected with 50 μL (1.75 MBq) marker on right flank for IVIS-imaging of the fluorophore and PET-imaging of ⁶⁴Cu over time. All eight mice were PET/CT and IVIS scanned at 1 h, 4 h, 24 h and 48 h post injection, and three mice were IVIS scanned after 2 weeks, 3 weeks and 4 weeks. Five mice were euthanized after PET/CT scanning and organs were afterwards collected and counted for 120 seconds on a gamma counter (Wizard2, Perkin Elmer) Well counter.

PET-procedure: Mice were anaesthetized using sevoflurane, placed on heated bed for scan, and scanned with CT and subsequent PET data were acquired on a MicroPET Focus 120 (Siemens Medical Solutions, Malvern, Pa., USA). The voxel size was 0.866×0.866×0.796 mm³, and in the center field of view the resolution was 1.4 mm full width at half-maximum (fwhm). PET-protocol for ⁶⁴Cu with emission time of 5 min for time points 1h and 4h, and 10 minutes for 24h, and furthermore 20 minutes for 48h scan. Data were reconstructed with the maximum a posterior (MAP) reconstruction algorithm. For anatomical localization of activity, CT images were acquired with a dediactes small animal imaging system (NanoScan microSPECT/CT, Mediso, Budapest, Hungary). After data reconstruction, PET and CT images were fused using the Inveon Software (Siemens). The emission scans were corrected for random counts and dead time. The PET and CT images were used to identify regions of tracer uptake and to generate regions of interest (ROIs) that were applied to each scan separately. A region of interest was drawn around the gel and liver and kidney, and either % ID/gel or % ID/g was calculated.

IVIS-procedure: Fluorescence imaging was performed using a small animal bioluminescence and fluorescence scanner (IVIS, Lumina XR, Caliper Life Sciences, USA). Mice were anaesthetized using isoflurane, placed on heated plate for scan, and fluorescence (FLI) scanned. A binning of 2, exposure time of maximum 120 seconds and excitation and emission wavelength of Ex: 745 nm and Em: 810-875 nm were employed.

Well counting: After last PET scan time (48h), five mice were euthanized and organs collected for well count for biodistribution. The well counting protocol consisted of 120 seconds counting per organ sample, and the results were presented as average mean % injected dose/g (% ID/g)±SEM.

Marker volume: The marker volume was obtained by automated segmentation procedure based on a CT contrast cut-off of 250 HU.

Results:

Overall, the activity concentration in the marker was found in increase 7.5% over the first 48h with less than 1% accumulating in the liver (FIG. 10A). PET images also confirmed that the main part of the activity was present in the marker volume (FIG. 11). The marker volume was found to decrease 11% during the first 48h (FIG. 10B), which is caused by EtOH efflux from the marker. The reduction in volume also explains the increase in the marker activity concentration per volume. Well counting data (FIG. 10D) furthermore agrees with the obtained PET based biodistribution, although an even higher activity concentration was found in the marker.

The NIR fluorescence intensity from PC2 in the marker was found to be approximately constant over 4 weeks with a slight increase at the 24h and 48h timepoint (FIG. 10C). The latter may be explained by fluctuations in instrument performance or positioning of the animal in the scanner, and is not considered as an actual change in NIR emission intensity. FLI images also show constant NIR fluorescence intensity from the marker over 4 weeks (FIG. 11) indicating that photobleaching is not changing the performance of the marker over time.

The CT contrast (FIG. 11) of the marker was found to be constant over the first 48h and the marker was clearly visible having contrast levels equal to bony structures.

Conclusion:

In conclusion, the ⁶⁴Cu radiolabelled SAIB:xSAIB:EtOH:PC2 70:10:20:0.01 marker was visible in PET/CT and NIR and displayed high stability in all image modalities in terms of position, intensity and retention of PC2 dye and ⁶⁴Cu activity.

### Example 15: ⁶⁴Cu Radiolabelling of NIR Markers Containing SSIB-Cy7.5 Using Ionophores

In the current example, the versatility of the current platform technology is demonstrated by radiolabelling a NIR marker embedding a non-chelating fluorophore using an alternative strategy. In this procedure, the marker LOIB:xSAIB:EtOH:D&Cv2:SSIB-Cy7.5 (70:10:20:0.01:0.01) was radiolabelled by ⁶⁴Cu using the hydrophobic chelator 8-hydroxy-quinoline (8HQ), thereby creating a marker that is visible in PET/CT/NIR, that have colour and is visible by the eye, and that forms a solid which is palpable in surgical procedures.

Method:

Marker formulation preparation: The LOIB:xSAIB:EtOH:D&Cv2:SSI B-Cy7.5 (70:10:20:0.1:0.01) marker formulation was prepared as described in example 4.

⁶⁴Cu preparation: [⁶⁴Cu]CuCl₂ was prepared as describe in example 10.

Radiolabelling of the marker. A solution of 8HQ in ethanol (200 μL, 500 μM) and pure ethanol (300 μL) was mixed with dry [⁶⁴Cu]CuCl₂ (300 MBq) and stirred at room temperature, 400 rpm, for 18 hours. Ethanol solvent was evaporated at 50° C. using argon flow for 20 minutes. Then 1 mL of marker formulation (LOIB:xSAIB:EtOH:D&Cv2:SSIB-Cy7.5 70:10:20:0.1:0.01) was added to the dry film of ⁶⁴Cu(8HQ) and hereafter the formulation was stirred at 50° C., 400 rpm for 2 hours. The radiolabelled marker formulation (660 μL) was transferred to a new glass vial and the radioactivity was measured by dose calibrator (Comecer, VDC-505). Non-radioactive gel formulation (1.5 mL) was added to dilute the formulation to 20 MBq/mL. The final formulation was homogenized by further stirring at 50° C., 400 rpm for 20 minutes and vortexing. Animal model: A marker with the composition LOIB:xSAIB:EtOH:D&Cv2:SSIB-Cy7.5 70:10:20:0.1:0.01) was radiolabelled with 20MBq ⁶⁴Cu(8HQ)/mL. A group of four female Balb/C CT26 mice were anesthetized with sevoflurane. The right flank of the mice was clipped and aseptically prepared for subcutaneous injection and each mouse was injected subcutaneously with 50 μL of gel formulation (20MBq/mL) corresponding to an activity dose level of 1 MBq/mouse.

PET data were acquired on a dedicated small animal PET/CT scanner (Inveon, MicroPET Focus 120 (Siemens Medical Solutions, Malvern, 30 PA, USA). The voxel size was 0.866×0.866×0.796 mm³, and in the center field of view the resolution was 1.4 mm full width at half-maximum (fwhm). PET scans were acquired 10 min after injection of the gel, and again 1 h, 17h and 42h after injection. Data were reconstructed with the maximum a posterior (MAP) reconstruction algorithm. For anatomical localization of activity, CT images were acquired with a MicroCAT II 35 system (Siemens Medical solutions, Malvern, Pa., USA). After data reconstruction, PET and CT images were fused using the Inveon Software (Siemens). The emission scans were corrected for random counts and dead time. The PET and CT images were used to identify regions of tracer uptake and to generate regions of interest (ROIs) that were applied to each scan separately. A region of interest was drawn around the gel and within the borders of the liver and kidney, and either % ID/gel or % ID/g was calculated.

Marker volume: The marker volume was obtained by an automated segmentation procedure based on a CT contrast cut-off of 250 HU.

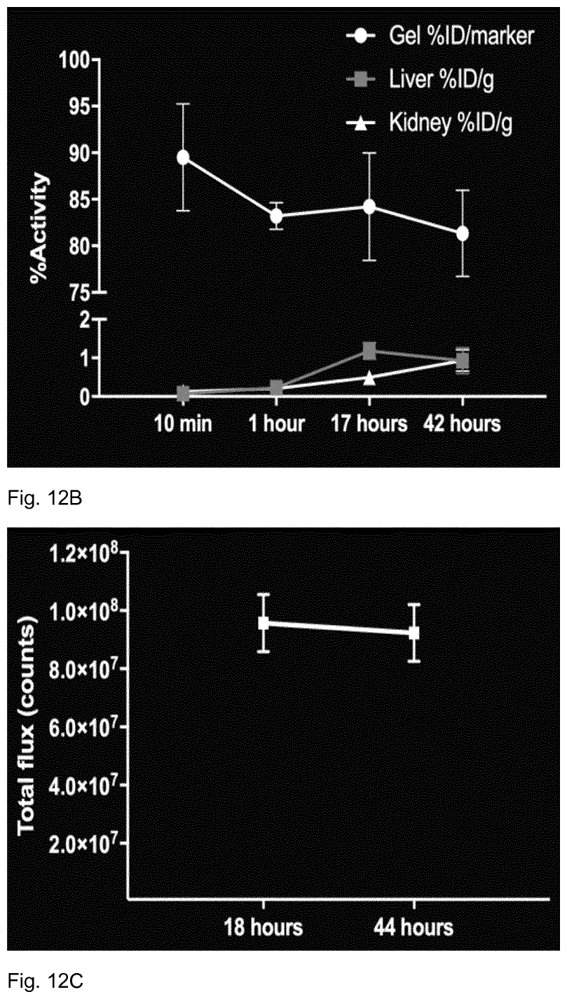

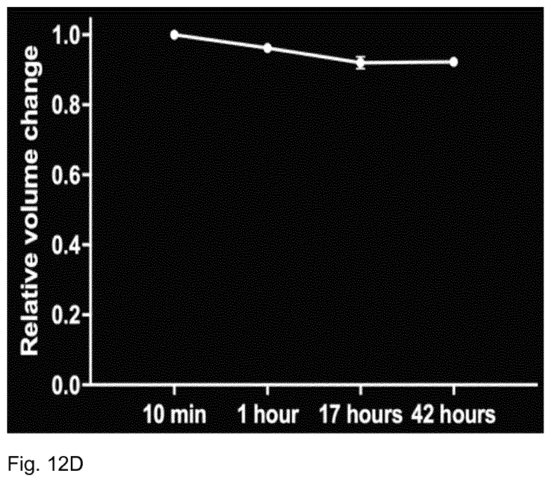

FLI images: Two mice were FLI imaged 18h and 44h post injection using the setup described in example 14. The total flux from the markers was subsequently integrated and presented in FIG. 12.

NIR camera images: A NIR camera (Fluobeam, 800 nm config, Fluoptics) was used to acquire NIR images.

Results:

The PET/CT/NIR data were collected and the results are presented in FIG. 12. A LOIB:xSAIB:EtOH:D&Cv2:SSIB-Cy7.5 70:10:20:0.1:0.01 marker formulation was successfully radiolabelled with ⁶⁴Cu complexed by 8HQ. The right flank of 4 female Balb/C mice was clipped and aseptically prepared for subcutaneous injection. The marker was injected subcutaneously and PET/CT images was recorded as function of time. At the initial scan-time (10 min) 90% of the total activity was recovered in the gel, which gradually declined to 81% after 42h (FIG. 12A). Over time, a minor fraction of ⁶⁴Cu was observed to accumulate in the liver and spleen, however less than 1.2% ID/g was found in both organs. The fluorescence intensity was found to be constant at 18h and 44h post injection, and the volume decreased 8% over the course of the experiment.

Conclusion:

In conclusion, 8HQ served as a hydrophobic ionophore for embedding ⁶⁴Cu into markers. Less than 10% decrease in activity was observed during 42h leading to minimal accumulation in the liver and spleen.

### Example 16: Radioiodine Labelling of NIR Markers Containing SSIB-Cy7.5 Using Ionophores

In the current example, the versatility of the current platform technology is demonstrated by radiolabelling a NIR marker embedding a non-chelating fluorophore using an alternative strategy. In this procedure, the marker LOIB:xSAIB:EtOH:D&Cv2:SSIB-Cy7.5 (70:10:20:0.01:0.01) was radiolabelled by ¹²⁵I using the SAIB-TMS substrate previously described (Schaarup-Jensen et. al. Injectable iodine-125 labeled tissue marker for radioactive localization of non-palpable breast lesions, Acta Biomaterialia 65 (2018), p. 197-202), thereby creating a marker that is visible in SPECT/CT/NIR, that have colour and is visible by the eye, and that forms a solid which is palpable in surgical procedures.

Methods:

TLCs were run in heptane:EtOAc (6:4) and developed with a KMnO₄ stain. SAIB-TMS eluted at Rf=0.6 and [¹²⁵I]SAIB-1 slightly below. Radio-TLCs were analyzed on a Cyclone Plus Storage Phosphor System (Perkin Elmer). Radioactivities were measured on a Veenstra Instruments dose calibrator VDC-505 in standardized 4 mL glass vials that had been pre-calibrated using the 1-125 specifications given by Perkin-Elmer.

Radioiodination: TI(CF₃COO)₃ (10.2 mg) was dissolved in a mixture of acetonitrile (2.30 mL) and trifluoroacetic acid (1.50 mL). An aliquot of this solution (380 μL) was transferred to an HPLC vial (TI(CF₃COO)₃: 1.1 mg, 1.8 μmol). To the vial was then added SAIB-TMS in acetonitrile (120 μL, 1.2 μmol). The solution was stirred at RT for 2 hours. To the mixture was then added [¹²⁵I]NaI in 10⁻⁵M aq. NaOH (30 μL, 96.5 MBq). After 43 minutes of stirring at RT, aq. NaI (18 μL, 3.6 μmol) was added, followed by 60 minutes of stirring at RT.

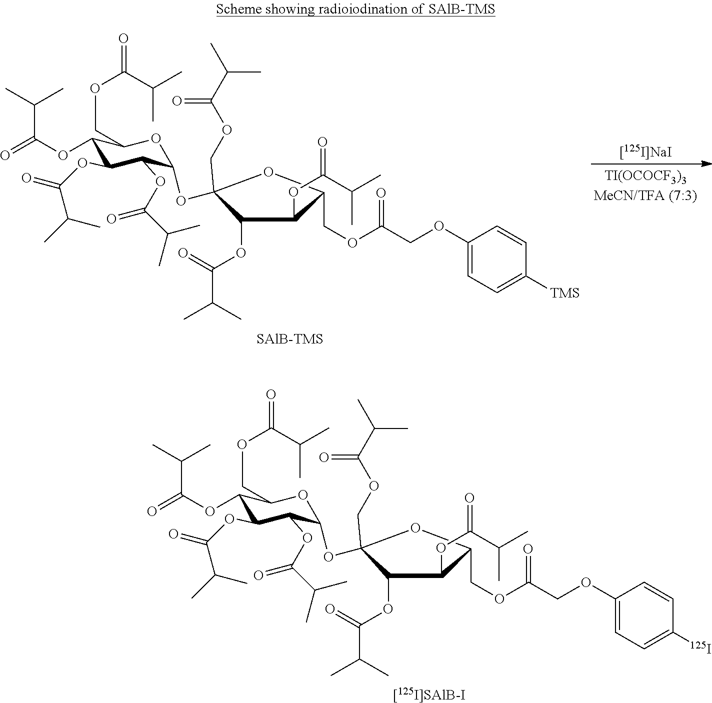

Work-up: To the reaction mixture was added water (500 μL). This was followed by capture of the product on a SEP-PEAK C18 Plus cartridge that had been pre-washed with ethanol (2×5 mL) and water (3×5 mL). The reaction vessel and cartridge were washed with aq. DTPA (3×1 mL, 50 mM, pH 7.0), water (3×10 mL) and 25% (v/v) ethanol in water (2×2 mL). The product was subsequently eluted in ethanol (3×1 mL). The two initial fractions (2×1 mL) of ethanol contained the bulk of the product. Radiochemical purity was determined in these two fractions by radio-TLC to be 90.6% (fraction 10, 51.6 MBq) and 92.6% (fraction 11, 30.0 MBq). The product was analysed by TLC and found to be chemically pure with the identity of the radiolabelled product confirmed by comparing with the Rf of non-radioactive SAIB-I. The two fractions were pooled with 500 μL extra ethanol added to rinse to containers. The radioactive yield was determined to be 83.4 MBq (RCY: 86%).

Formulation of marker: From the combined product fractions in ethanol (2.5 mL) was removed 2.2 mL (73 MBq), which was evaporated to dryness in a glass vial for 74 minutes at 40° C. under at stream of argon. To the dry residue was added 1.8 mL LOIB:xSAIB:EtOH:D&Cv2:SSIB-Cy7.5 marker solution, followed by magnetic stirring at RT for 30 minutes. This gave the finished marker formulation.

In vitro retention of radioactivity: Approximately 3×100 μL of the gel solution containing the radiolabel was transferred to glass vials containing Dulbecco's phosphate buffered saline (3.0 mL). At several time points, 2 mL of the medium was removed and replaced with 2 mL fresh medium. The radioactivity left in the vial was measured after this replacement and is shown in Table 1. In the last displayed measurement point (X), the entire medium was removed and replaced with fresh medium. This was done immediately after the final 10 day measurement. The radioactivity in the removed medium aliquots was also measured and is shown in Table 3.

Animal model: The right flank of female NMRI mice or 12-week-old female Balb/C mice was clipped and aseptically prepared for subcutaneous injection of the marker. After the injection of the markers, SPECT/CT scanning and fluorescence imaging was performed.

SPECT/CT scanning: microSPECT/CT scans of ¹²⁵I-rad iolabeled LOIB:xSAlB:EtOH:D&Cv2:SSIB-Cy7.5 marker injected mice were performed using a dedicated small animal SPECT/CT scanner (NanoScan, Mediso, Budapest, Hungary). SPECT scans were performed as a single field of view (FOV) over the area injected with the marker. Scans were performed using a micro pin-hole collimator and 20 projections of 90 seconds were acquired for all scans.

On SPECT/CT images a manual volumes of interest (V01) was constructed using commercially available software (Vivo-quant 3.5, inviCRO LCC. Boston, Mass., USA). Decay corrected ¹²⁵I-activity was calculated for scan points to determine ¹²⁵I retention in the marker.

FLI images: Fluorescence imaging of the marker was performed using a small animal fluorescence and bioluminescence imaging (FLI) and X-ray system (IVIS Lumina XR, Caliper Life Sciences, USA) using the settings described in example 14. FLI/X-ray imaging (ex/em, light, 10 cm FOV) was performed on the day of injection and weekly for three weeks after injection of the marker. Corresponding radiographs and light images were recorded. Images were evaluated for emission yield by manually constructing a ROI three times larger than the area of the gel and recording total flux (counts/sec) in the constructed ROI.

Results:

TABLE 3
¹²⁵I retained in the marker and release ¹²⁵I in the media as function of time.

| Time | | | % in removed | | Accumulated | |
|---|---|---|---|---|---|---|
| (days): | Retention | SD | medium | SD | release | SD |
| 0.00 | 100% | 0 | 0.00% | 0.00% | 0.00% | 0.00% |
| 0.06 | 97.00% | 2.20% | 0.04% | 0.02% | 0.04% | 0.02% |
| 0.88 | 95.50% | 1.80% | 0.13% | 0.09% | 0.17% | 0.08% |
| 3.02 | 94.70% | 2.60% | 0.24% | 0.08% | 0.40% | 0.16% |
| 6.10 | 94.20% | 2.50% | 0.20% | 0.07% | 0.61% | 0.20% |
| 10.06 | 94.00% | 3.00% | 0.20% | 0.06% | 0.81% | 0.23% |
| X | 95.50% | 0.50% | 0.16% | 0.05% | 0.97% | 0.26% |

An excellent retention of radioactivity was observed in the markers, with measured radioactivities in the gel dropping during the first day of measurement to about 95%, reaching a slowly declining plateau. In the final measurement point after 10 days of incubation (X), where only the radioactivity in the marker was measured, the retention was 95.5±0.5% (n=3). The initial drop in retained radioactivity could be attributed to burst release but since it was not mirrored by the radioactivity in the released medium, it is likely that the initially observed drop is a consequence of the geometric shape of the gel changing as ethanol is released and the gel settles. During the initial day of monitoring, a release of only 0.17±0.08% (n=3) was observed in the removed medium, with a slow and steady increase to about 1% released in total over the consecutive 10 days.

Conclusion:

In conclusion, these data underscore a very high degree of 1-125 radioactivity retention in this LOIB:xSAlB:EtOH:D&Cv2:SSIB-Cy7.5 marker system.

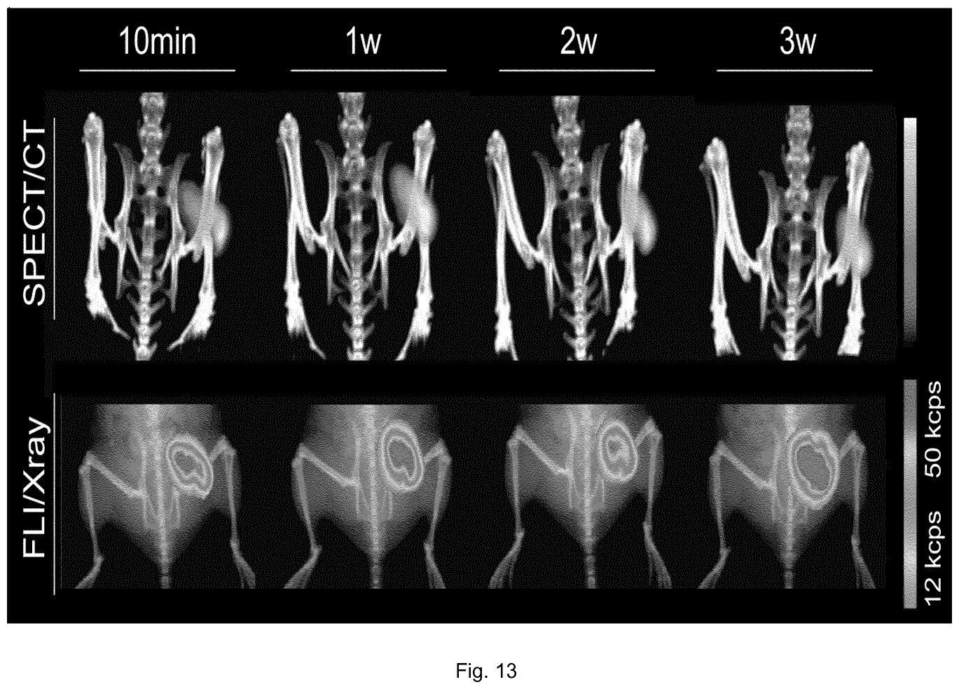

The SPECT/CT and FLI/Xray images (FIG. 13) furthermore confirms that the ¹²⁵I radiolabelled LOIB:xSAIB:EtOH:D&Cv2:SSIB-Cy7.5 markers were highly stable with respect to position, intensity and retention of dye and ¹²⁵I activity over a 3 week period.

1. A solution comprising
a. a water insoluble carbohydrate,
b. a fluorescent dye, and
c. an organic solvent having a log P in the range of −2 to 2.

2. The solution according to claim 1, wherein the fluorescent dye has a log P above 2, thereby providing retention of the fluorescent dye in the solution under aqueous conditions.

3. The solution according to claim 1, wherein less than 10% of the fluorescent dye is released after 5h under aqueous conditions, such as less than 5%.

4. The solution according to claim 1, wherein the fluorescent dye is a near infrared (NIR) contrast agent.

5. The solution according to claim 1, wherein NIR contrast agent is selected from the group consisting of phthalocyanines, naphthalocyanines, porphines, antracocyanine and cyanine dyes.

6. The solution according to claim 1, wherein NIR contrast agent is a phthalocyanine, such as PC1, PC2 and/or PC3.

7. The solution according to claim 1, wherein the fluorescent dye is conjugated to a water insoluble carbohydrate selected from the group consisting of SAIB, SSIB and LOIB.

8. The solution according to claim 1, wherein the water insoluble carbohydrate comprises a disaccharide selected from the group consisting of maltose, trehalose, lactose and sucrose, wherein one or more hydroxyl groups are functionalized to render the carbohydrate water insoluble.

9. The solution according to claim 1, wherein the water insoluble carbohydrate comprises one or more hydroxyl groups functionalized to form C2-C7 esters.

10. The solution according to claim 1, wherein the number of hydroxyl groups functionalized to form C2-C7 esters is n, n-1, n-2, n-3, n-4 or n-5, wherein n is the total number of hydroxyl groups of the carbohydrate.

11. The solution according to claim 1, wherein the C2-C7 esters are formed by a bond between the hydroxyl group(s) of the carbohydrate and the carbonyl group of a C2-C7 alkanoyl(s).

12. The solution according to claim 1, wherein the C2-C7 alkanoyl is selected from acetyl, propanoyl, isobutanoyl and benzoyl.

13. The solution according to claim 1, wherein the water insoluble carbohydrate is selected from the group consisting of maltose octaisobutyrate (MOIB), sucrose diacetate hexaisobutyrate (SAIB), sucrose octaisobutyrate (SOIB), lactose octaisobutyrate (LOIB), and trehalose octaisobutyrate (TOIB).

14. The solution according to claim 1, wherein the organic solvent diffuses out of the solution under aqueous conditions, providing a gel, a glass, a semi-solid, a solid, a crystal or any combination thereof.

15. The solution according to claim 1, wherein the viscosity increases by more than 1000 centipose (cP) under aqueous conditions, such as more than 5000 cP, for example more than 10000, such as more than 50000 cP, for example more than 100000 cP.

16. The solution according to claim 1, wherein the solution sets under aqueous conditions in less than 5 h, such as less than 4 h, for example less than 3 h, such as less than 2 h.

17. The solution according to claim 1, wherein the aqueous conditions are in vivo conditions.

18. The solution according to claim 1, wherein the organic solvent is selected from the list consisting of methanol, ethanol, propanol, isopropanol, butanol, isobutanol, tert-butanol, benzyl alcohol, propylene carbonate and dimethyl sulfoxide.

19. The solution according to claim 1, further comprising a monoglyceride, diglyceride and/or triglyceride.

20. The solution according to claim 1, wherein the fluorescent dye is coordinated to a radionuclide.

21. The solution according to claim 1, wherein the radionuclide is selected from the group consisting of Tc-99m, In-111, Ga-67, Lu-177, Tl-201, Sn-117m, Cu-64, Mn-52, Zr-89, Co-55, Sc-44, Ti-45, Sc-43, Cu-61, As-72, Te-152, F-18, Ga-68, C-11, Nd-140 and Te-149.

22. The solution according to claim 1, comprising Cu-64 and PC1, PC2, and/or PC3.

23. The solution according to claim 1, comprising a further imaging agent.

24. The solution according to claim 1, wherein the further imaging agent is selected from the group consisting of X-ray agent, CT agent, MRI agent, PET agent and SPECT agent.

25. The solution according to claim 1, wherein the further imaging agent has a structure according to formula (III),

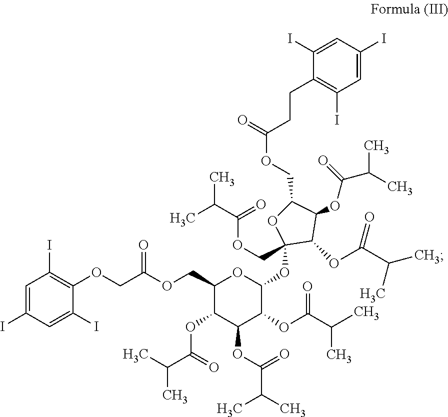 [SMILES: CC(C)C(=O)OC[C@@]1(O[C@H]2O[C@H](COC(=O)COc3c(I)cc(I)cc3I)[C@@H](OC(=O)C(C)C)[C@H](OC(=O)C(C)C)[C@H]2OC(=O)C(C)C)O[C@H](COC(=O)CCc2c(I)cc(I)cc2I)[C@@H](OC(=O)C(C)C)[C@@H]1OC(=O)C(C)C]

26. A solution according to claim 1 for use as an in vivo diagnostics tool.

27. A method of in vivo imaging, the method comprising
a. administering a solution according to any one of the preceding claims to an individual in need thereof,
b. excitation of the fluorescent dye, and
c. detection of the fluorescent dye.

28. The method according to claim 27, wherein the solution is administered by injection and/or smearing.

29. A method involving using the solution according to claim 1 for in vivo imaging.

30. A method involving using the solution according claim 1 as a fiducial marker.

31. A method involving using the solution according to claim 1 for guidance of surgery.
